(19) United States
(12) Patent Application Publication
(10) Pub. No.: US 2024/0274226 A1
(43) Pub. Date: Aug. 15, 2024

(54) MOLECULAR EVALUATION METHODS

(71) Applicant: UNIVERSITY COLLEGE DUBLIN, Dublin (IE)

(72) Inventors: Oleksii RUKHLENKO, Dublin (IE); Vadim ZHERNOVKOV, Dublin (IE); Walter KOLCH, Dublin (IE); Boris KHOLODENKO, Dublin (IE)

(21) Appl. No.: 18/564,440

(22) PCT Filed: May 27, 2022
(86) PCT No.: PCT/EP2022/064502

§ 371 Date: Nov. 27, 2023

(30) Foreign Application Priority Data
May 27, 2021 (GB) 2107576.7

(51) Int. Cl. G16B 5/20; G16B 20/00; G16B 40/20

(52) U.S. Cl. CPC G16B 5/20; G16B 20/00; G16B 40/20

(57) ABSTRACT

A molecular evaluation method for predicting the molecular mechanisms through which a perturbation promotes or inhibits a cellular transition from a first cell state to a second cell state. Cells are clustered in a multi-dimensional representation to identify distinct cell states, with dimensions corresponding to molecular features, which are ranked according to a vector component directed towards a separating hypersurface. Core network components are identified with the highest ranking and a reduced dimension space is used, without the core component dimensions, to assess the effects of perturbations in terms of a Dynamic Phenotype Descriptor (DPD) which represents the remainder of the global network on which the core network acts. Bayesian Modular Response Analysis is used to reconstruct the topology and signs and strengths of causal connections between nodes of the core network and the DPD. A resulting mechanistic model based on ordinary differential equations (ODE) is derived that calculates the quality and quantity of changes which are needed to convert one cell state into another permitting interventions to be identified that will promote or inhibit particular cell transitions.

### TECHNICAL FIELD

This invention relates to molecular evaluation methods, and in particular to methods for predicting the molecular mechanisms through which a perturbation promotes or inhibits a cellular transition from a first cell state to a second or more other cell states.

### BACKGROUND ART

The concept of cell states has a long tradition in biology. Cell states help describe how biological processes combine to form autonomous units with defined form and function. The cell state concept has proven a very useful lens to view and understand the organization of tissues and organisms, their development and responses to exogenous and endogenous changes in health and disease.

While initially mainly based on descriptions of morphology and phenotypes, progress in global analysis methods now allows phenotypes to be connected with underlying molecular processes. These connections enable the characterization of cell states with fine molecular resolution and open the door to understand how cell states can evolve and transition into each other.

In 1940 Waddington suggested that cells move through a landscape of mountains and valleys as rolling marbles from one (meta)stable state to another (Organisers and Genes by C. H. Waddington, The University Press, 1940). This now famous model appeals through its intuitive nature but leaves open how and why the marbles roll into certain valleys and also whether they can go back to an initial state.

Recent efforts have applied computational models to understand transitions between states. These include models that describe cell state transitions as static states generated through convergence of molecular processes or dynamic states created by stochastic transitions (Brackstn, R. D. et al. (2018). Transition state characteristics during cell differentiation. PLOS Comput Biol 14, e1006405; Wang, J. et al. (2011). Quantifying the Waddington landscape and biological paths for development and differentiation. Proc Natl Acad Sci USA 108, 8257-8262).

Other models use lineage analysis of cell states to characterize and infer cell state transitions (Hormoz, S. et al. (2016). Inferring Cell-State Transition Dynamics from Lineage Trees and Endpoint Single-Cell Measurements. Cell Syst 3, 419-433 e418; Su, Y. et al. (2020). Multi-omic single-cell snapshots reveal multiple independent trajectories to drug tolerance in a melanoma cell line. Nat Commun 11, 2345). These efforts show that cell states are interconvertible and that this involves changes in dynamic molecular processes, such as gene expression and signal transduction networks. However, a critical gap is the lack of a mechanistic understanding of how cellular networks drive cell state transitions that would allow us to purposefully manipulate and control cell states.

US 2008/0195322 A1 discloses a method for profiling the effects of perturbations on biological samples by acquiring images of cells in different cell states, applying statistical multivariate methods that use morphological features derived from the images to separate the cell states, and distinguish morphological changes in response to different perturbations, such as drug treatments. The method provides no predictive value in designing perturbations to achieve a desired cell state and nor does it provide any insight into the cause of the morphological changes observed. It simply screens compounds according to the observed effect on cells. No information is generated regarding the governing molecular networks involved in cell state transitions, rendering it unsuitable for predicting molecular mechanisms.

### DISCLOSURE OF THE INVENTION

There is provided a molecular evaluation method for predicting the molecular mechanisms through which a perturbation promotes or inhibits a cellular transition from a first cell state to a second cell state, comprising the steps of:
a. processing data for a population of cells to identify cells associated with said first and second states respectively, wherein said processing comprises (i) mapping said cells in a multi-dimensional space whose dimensions correspond to distinct molecular features of the cells that define said cell states, the molecular features being selected from RNA expression, protein expression and posttranslational protein modification, and (ii) identifying clusters of cells in said representation associated with said first and second states;
b. constructing a hypersurface in said representation that separates said first and second states;
c. generating a state transition vector (STV) of unit length that determines the direction from the first state to the second state in said representation;
d. generating a ranked list of said molecular features, wherein each molecular feature is ranked by determining the magnitude of the STV component in a dimension associated with the molecular feature;
e. identifying within said ranked list the components of a core biochemical network comprising the top ranked components, or the components regulating the top ranked components, above a cut-off in the ranking;
f. calculating, in a reduced multi-dimensional space whose dimensions correspond to those molecular features not identified as components of the core biochemical network, a dimensionality-reduced representation of said hypersurface and a dimensionality-reduced STV;
g. determining the effect of a perturbation by generating a perturbation vector in the reduced multi-dimensional space, the perturbation vector connecting the centroids of point clouds associated with cell states before and after the perturbation was applied to cells;
h. classifying the perturbation as promoting a transition between said first and second cell states when the dot product of the perturbation vector and the dimensionality-reduced STV is positive or inhibiting said transition when said dot product is negative;
i. defining a Dynamic Phenotype Descriptor (DPD) that quantifies cell phenotypic changes in response to a perturbation by measuring whether the perturbation moves the cell states towards or away from the dimensionality-reduced hypersurface, wherein the DPD comprises the molecular features in the ranked list which are not components of the core biochemical network; and
j. calculating a respective causal network graph for each of the first and second cell states, wherein each causal network graph is a directed, weighted network graph having the same nodes, the nodes of each graph comprising each of the core components together with the DPD represented as a node, and each causal network graph having directed and weighted edges that are specific to the associated first or second cell state, whereby each of said graphs quantifies the effects of the core components on the DPD in the first or second cell state respectively and thereby describes the molecular mechanisms that characterise the first and second cell states.

In determining the ranked list of molecular features and the top ranked components, the list and/or ranking may be limited to actionable components, i.e. enzymes, transcription factors, transporters, channels, receptors, scaffolds, and the like. In this way the ranked list may exclude high-rank components that do not affect other proteins and cannot be inhibited (e.g. some structural proteins, for instance, caveolin).

The term “perturbation” encompasses (where the context permits) a combination of individual perturbations.

The cell states referred to as “first” or “second” cell states are merely two possible cell states which can be adopted, and does not imply that the system has only two such states. The claimed method may encompass any other number of cell states and may generate network graphs for each such cell state quantifying the effects of the core components on the DPD in each such cell state to describe the molecular mechanisms that characterise those cell states.

The term “graph” does not imply any specific graphical representation, but rather denotes a mathematical graph i.e. a structure which models pairwise relations between the core network components and DPD (i.e. the nodes) in terms of connection strengths (i.e. the weighted, directed edges). The method is typically a computer-implemented method embodied in program instructions which when executed on a suitable computing system cause the method to be carried out by that computing system.

Preferably, the step of calculating a respective causal network graph comprises calculating a respective causal network connection matrix specifying the strength of connection between each of the core components and between each core component and the DPD.

Preferably, the calculation of a causal network connection matrix comprises inferring the topology and strengths of causal connections of the core network and the DPD using Modular Response Analysis.

More preferably, the Modular Response Analysis used is Bayesian Modular Response Analysis.

Preferably, the method further comprises the steps of experimentally perturbing the cell states, observing the effect of the perturbation on the cell states, and inferring from the observed effects the strength of connection between each of the core components, and between each core component and the DPD.

Preferably, observing the effect of the perturbation on the cell states comprises measuring one or more molecular responses to the experimental perturbation.

Preferably, experimentally perturbing the cell states comprises applying a plurality of perturbations and observing the effect of the perturbations on the cell states.

Preferably, a perturbation comprises exposing cells to a chemical compound, exposing cells to a biological compound, inducing an epigenetic or genetic change in cells, exposing cells to pathogens, exposing cells to an interaction with other cells, and exposing cells to an interaction with a biological or artificial surface.

Further, preferably:
(i) the perturbation comprises exposing cells to a chemical compound, the chemical compound comprising a pharmaceutical drug, a toxin, or an environmental chemical;
(ii) the perturbation comprises exposing cells to a biological compound, the biological compound comprising a growth factor, a hormone, a cytokine, a toxin, an antibody, a cellular receptor, an siRNA, an shRNA, or a ligand;
(iii) the perturbation comprises exposing cells to an epigenetic or genetic change comprising an epigenetic modification, a gene mutation, a heterozygote gene knockout, a gene copy number aberration, a gene structural variant, or CRISPR/Cas9-mediated genetic modification; or
(iv) the perturbation comprises exposing cells to a pathogen comprising a virus or a bacterium.

Preferably, step (g) comprises processing data for a population of cells to which the or each perturbation has been applied, wherein said processing comprises (i) mapping said cells in said reduced multi-dimensional space, and (ii) identifying clusters of cells in said mapped cells associated with the cells before and after the perturbation is applied.

Preferably, identifying within said ranked list the components of a core biochemical network comprising the top ranked components above a cut-off in the ranking, comprises determining a cut-off in the ranking which maximises the number of components which can be mapped onto existing biochemical pathways while minimising the total number of ranked components used according to an optimisation function.

Further, preferably, determining the number of components which can be mapped onto existing biochemical pathways comprises determining from one or more databases whether each component can be mapped to a pathway whose characteristics are known from the one or more databases.

The method may be adapted to multi-state cellular transitions by applying the method to evaluate the transitions between different pairs of a multi-state system.

In some embodiments, therefore, said first and second cell states are any two states chosen from a set of three or more cell states, and the step of processing data for a population of cells identifies cells associated with said three or more cell states by identifying clusters of cells in said representation associated with each of said three or more cell states.

Preferably, said hypersurface is a hyperplane.

Preferably, said distinct molecular features of the cells are identified in said processed data as a set of measured analyte levels each of which corresponds to a distinct molecular feature.

There is also provided a molecular evaluation method according to any preceding statement of invention, wherein said molecular features of the cells that define said cell states are selected from RNA expression, protein expression and posttranslational protein modification.

The method preferably further comprises identifying an intervention likely to promote or inhibit a cellular transition between first and second cell states, by one or more of:
a. using the causal network graphs to identify an intervention that will change one cell state into another cell state; or
b. assessing by in silico simulations of kinetic computational models developed on the basis of said causal network graphs whether an intervention will move a said cell state along the STV away from, towards or across the separating hypersurface.

Suitably, the intervention is a combination of interventions, and the assessment in step (a) or (b) considers the effect of the interventions simultaneously.

Alternatively, the intervention is a combination of interventions, and the assessment in step (a) or (b) considers the effect of the interventions serially.

In certain embodiments, determining whether an intervention will change one cell state into another cell state comprises determining whether the distance from the first cell state data points to the hypersurface decreases following said intervention.

In other embodiments, determining whether an intervention will move a said cell state along the STV away from, towards or across the separating hypersurface comprises calculating a change in the DPD using a computational model built from the data, as given by:

$$\frac{dS}{dt} = f ( S ) + {\sum }_{j} {r}_{s j} ( \frac{{S}_{st . st .}}{{x}_{j}^{st . st .}} ) {x}_{j} ( t )$$

wherein S is the DPD value being calculated by the model, ƒ(S) is the restoring driving force,

$${\sum }_{j} {r}_{s j} ( \frac{{S}_{st . st .}}{{x}_{j}^{st . st .}} ) {x}_{j} ( t )$$

is the signaling driving force, xⱼ(t) are the outputs of signaling modules, r_{Sj} are the corresponding, BMRA-inferred connection coefficients to the STV (see Table S4), and S_{st.st.} and xⱼ^{st.st} are the initial steady-state values of S and x; before perturbations.

The methods of the invention may be implemented as a system, a method, and/or a computer program product at any technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Python, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein as method steps. It will be understood that each step, and combinations of steps, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

### BRIEF DESCRIPTION OF THE DRAWINGS

The invention will now be further illustrated by the following description of embodiments thereof, given by way of example only with reference to the accompanying drawings, in which:

FIG. 1 is a schematic overview of the method steps;

FIG. 2 shows how activation of different receptor kinases leads to different cell fates;

FIG. 3 is a plot of the average number of neurites per cell in different cell populations;

FIG. 4 is a plot of PCA compressed RPPA data for TrkA and TrkB cells in the space of the first two principal components that are normalized by the data variance captured by these components;

FIG. 5 is a plot of PCA compressed RPPA data for TrkA and TrkB cells after growth factor stimulation in the space of the first three principal components, showing the separation hyperplane;

FIG. 6 is a schematic illustration of the influence of core network components with respect to the global signaling network and the influence on cell fates;

FIG. 7 is a plot similar to that of FIG. 5, but showing centroids of data clouds only, and adding the effect of a perturbation, and also showing the decomposition of a perturbation vector into a vector that is collinear with the STV and a vector perpendicular to the STV;

FIG. 8 is a prior topology of core network connections based on existing knowledge;

FIG. 9 is a series of Western Blots illustrating the time courses of pTRK and ppERK activation in TrkA and TrkB cells after stimulation with 100 ng/ml NGF or BDNF, respectively;

FIGS. 10 and 11 show the inferred core signaling network topologies for TrkA and TrkB cells, reconstructed by BMRA;

FIGS. 12 and 13 show the inferred signaling network topologies for TrkA and TrkB reconstructed by BMRA and including the DPD node;

FIGS. 14 and 15 plot PCA-compressed 45 min data points for TrkA cells, TrkB cells, and (in FIG. 15) TrkB cells treated with p90RSK inhibitor, BI-D1870. The distances of centroids of TrkB cells to the separation surface (grey) before and after perturbation are shown by black lines;

FIG. 16 is a plot of the restoring driving force against the DPD output;

FIG. 17 is a plot of the corresponding Waddington's landscape potential against the DPD output;

FIGS. 18-24 show experimental data points plotted over model-predicted time courses for TrkA and TrkB cells simulated with MGF and BDNF, respectively;

FIGS. 25-30 show experimental data points plotted over model-predicted time courses for TrkA and TrkB cells treated with various different inhibitors;

FIG. 31 is a plot of experimental data points plotted over model-predicted time courses showing the DPD output S response to ligand stimulation in TrkA and TrkB cells;

FIGS. 32 and 33 are live cell images of TrkA and TrkB cells respectively, stimulated with GFs for 72 hours;

FIG. 34 shows model-predicted time courses and experimentally measured DPD responses of TrkA (grey) and TrkB (black) cells to diverse inhibitor perturbations;

FIG. 35 is a set of live cell images taken at 72 hours in TrkA and TrkB cells exposed to various perturbations, accompanied by bar plots showing the percentage of differentiated cells for certain of the images;

FIG. 36 is a plot of the simulated DPD time course of NGF-stimulated TrkA cell response with and without AKT activation;

FIGS. 37 and 38 are live cell images of TrkA cells transfected with myristoylated AKT 72 before and after stimulation with NGF for 72 hours;

FIG. 39 is a set of live cell images taken at 72 hours in TrkA cells stimulated with NGF and treated with various inhibitors;

FIG. 40 is a set of live cell images taken at 72 hours in TrkB cells stimulated with BDNF and treated with various inhibitors;

FIG. 41 is a predictive simulation of DPD responses of TrkB cells to ERBB and ERK inhibitors applied separately and in combinations shown at 45 min 100 ng/ml BDNF stimulation, illustrated using Loewe isoboles;

FIG. 42 is a predictive simulation of DPD responses of TrkA cells to ERBB and ERK inhibitors applied separately and in combinations shown at 45 min 100 ng/ml NGF stimulation, illustrated using Loewe isoboles;

FIG. 43 shows responses of ERK (ppERK) and p70S6K (pS6K) phosphorylation to Geftitinib (ERBB family inhibitor, applied at 5 and 10 μM), Trametinib (MEK inhibitor, 1 and 2 μM) and their combination (2.5 μM and 0.5 μM) at 45 min in TrkB cells;

FIG. 44 shows responses of FAK phosphorylation to Geftitinib (2.5 and 5 μM), Trametinib (0.1 and 0.2 μM) and their combination (2.5 and 0.05 μM, and 1.25 and 0.1 μM) at 72 hours;

FIG. 45 is a live cell image of TrkB cells stimulated with BDNF taken at 72 hours;

FIG. 46 is a live cell image of BDNF-stimulated TrkB cells treated with 0.2 μM Trametinib taken at 72 hours;

FIG. 47 is a live cell image of BDNF-stimulated TrkB cells treated with 2.5 μM Gefitinib taken at 72 hours;

FIG. 48 is a live cell image of BDNF-stimulated TrkB cells treated with a combination of 1.25 μM Gefitinib and 0.1 μM Trametinib taken at 72 hours;

FIG. 49 is a live cell image of BDNF-stimulated TrkB cells treated with a combination of 2.5 μM Gefitinib and 0.05 μM Trametinib taken at 72 hours;

FIGS. 50 and 51 are live cell images of NGF-stimulated TrkA treated with a combination of 1.52 μM Geftitinib and 0.1 μM Trametinib taken at 72 hours;

FIG. 52 is a bar plot of the percentage of differentiated cells observed for different treatments.;

FIG. 53 is a 3D plot showing the separation of MS phosphoproteomic patterns of TrkA and TrkB cell states and the STV projection into the PCA space;

FIG. 54 is a bar chart showing DPD values calculated using MS phosphoproteomics data for TrkA and TrkB cells treated with Trametinib (0.5 μM), Gefitinib (2.5 μM), and their combination (0.25 μM and 1.25 μM) at 45-minute stimulation;

FIG. 55 is a 2D plot showing the separation of apoptotic and proliferation states of SKMEL-133 cells and a projection into the PCA space;

FIG. 56 is an inferred topology of a core signaling network for the SKMEL-133 cells;

FIG. 57 is an inferred topology of a core signaling network for the SKMEL-133 cells with addition of c-MYC;

FIGS. 58-63 show the model calculated and experimentally determined DPD responses of SKMEL-133 cells to MEK, AKT, PKC, SRC, mTOR and CDK, respectively;

FIGS. 64-66 illustrate model-predicted SKMEL-133 cell maneuvering in Waddington's landscape following inhibitor treatments applied separately and in combination.

### DETAILED DESCRIPTION OF PREFERRED EMBODIMENTS

In the following description there is disclosed a cell State Transition Identification and Control Key (cSTAR) that identifies cell states, quantifies their determining elements, reconstructs a mechanistic network that controls the cell state transitions, and unlocks pathway manipulations that allow us to convert one cell state into another.

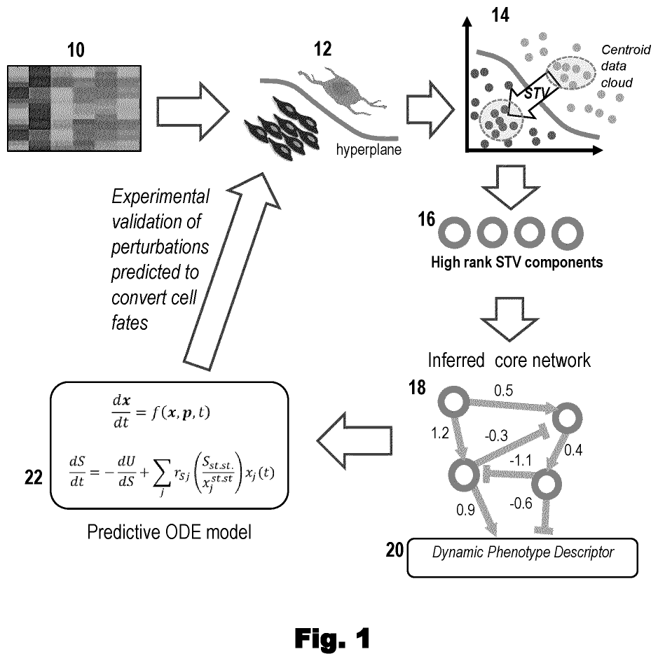

The overview of the method is shown in FIG. 1. cSTAR uses molecular data sets as input, step 10, that contain enough information to distinguish different cell states. Typically, this will be omics data. Here, we have used RPPA derived phosphoproteomics data, but other omics data are also suitable provided that they can reflect perturbations that change cell states.

cSTAR sequentially integrates the following elements:
clustering of the measured data and construction of a hyperplane that separates the molecular features which characterize a cell state, step 12. We use support vector machines (SVMs), as they exploit high dimensional space to efficiently separate data by maximizing the distance between data points belonging to different cell states;
a State Transition Vector (STV) that in the molecular dataspace of the input data indicates a path from the centroid of a point cloud of one cell state to the centroid of a point cloud of another cell state, step 14. The STV identifies the components 16 of the core signaling network 18 that governs the transition between two cell states;
a Dynamic Phenotype Descriptor (DPD) 20 that quantifies cell phenotypic changes in response to a perturbation by measuring whether the perturbation moves the centroid that characterizes a particular cell state towards or away from the hyperplane;
a Bayesian formulation of Modular Response Analysis (BMRA) (Kholodenko et al., (2002). Untangling the wires: A strategy to trace functional interactions in signaling and gene networks. Proceedings of the National Academy of Sciences 99, 12841; Halasz, M. et al. (2016) Integrating network reconstruction with mechanistic modeling to predict cancer therapies. Sci Signal 9, ra114), which reconstructs the topology and signs and strengths of causal connections between nodes of the core network created from the components specified by the STV. The DPD 18 is an additional node in this core network representing the remainder of the global network upon which the core network acts to drive cell fate transitions; and
a resulting mechanistic kinetic model based on ordinary differential equations (ODE) that calculates the quality and quantity of changes which are needed to convert one cell state into another. This model gives a mathematical description of the forces that move a cell along Waddington's landscape and provides direct instructions for experimental perturbations that can convert one cell state into another.

### Experimental System and Datasets

The cSTAR approach was validated experimentally, and the method used to devise specific drug perturbations to cell signaling networks that successfully converted proliferation into differentiation states in the neuroblastoma SH-SY5Y cell line. While here we used reversed phase protein arrays (RPPA) as data source, cSTAR is a versatile method that can utilize different types of omics data to design precision interventions for controlling and interconverting cell fate decisions.

In order to rigorously test cSTAR we were looking for an experimental system that is a meaningful biological model and features robust cell fate decisions based on subtle molecular differences. This poses a stringent challenge of distinguishing cell states and a realistic chance of identifying feasible perturbations that can convert cell fates. The SH-SY5Y human neuroblastoma cell line is a well-established cell model for studying neuronal differentiation, neurodegeneration, and therapeutic target discovery in neuroblastoma.

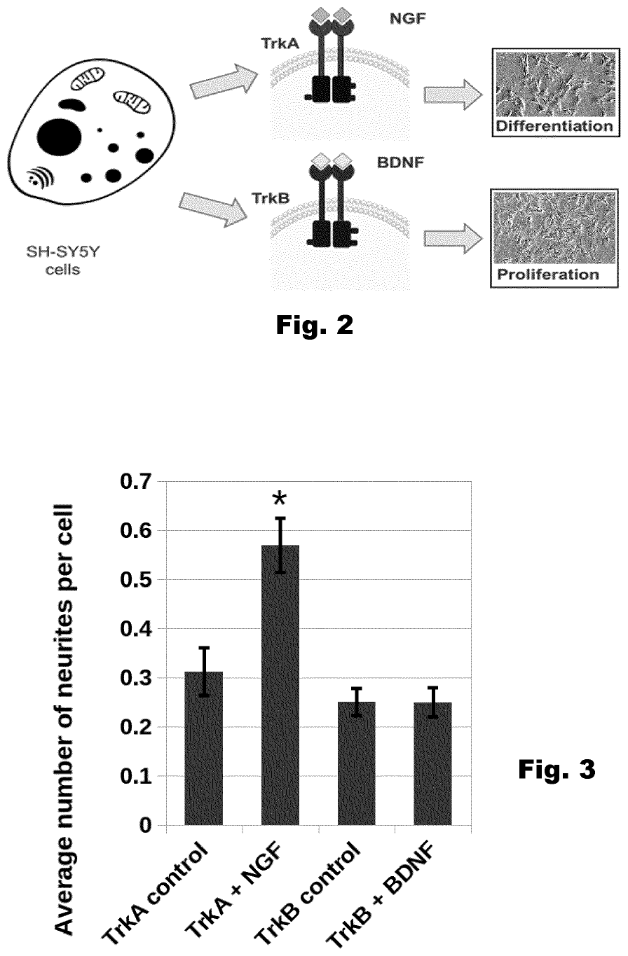

Expression of the TrkA or TrkB receptor tyrosine kinases specifies different cell fate decisions in SH-SY5Y cells. TrkA stimulates terminal differentiation marked by neurite outgrowth, whereas TrkB drives proliferation, as illustrated in FIG. 2. These diverse phenotypes correlate with clinical outcomes in neuroblastoma.

FIG. 2 shows how activation of TrkA cells by NGF leads to differentiation, whereas activation of TrkB cells by BDNF leads of proliferation. SH-SY5Y cells stably expressing TrkA or TrkB receptors were stimulated with 100 ng/ml NGF or BDNF, respectively. Differentiation and proliferation were assessed 72 hours after growth factor treatment.

FIG. 3 shows a plot of the quantification of the average number of neurites per cell. Neurite outgrowth is a hallmark of cell differentiation.

TrkA expression is associated with good prognosis, while TrkB expression correlates with aggressive tumor behavior. TrkA and TrkB activate very similar signaling pathways, and it is unclear what particular changes in signaling and expression patterns cause these distinct cell fate decisions (Schramm, A. et al. (2005). Biological effects of TrkA and TrkB receptor signaling in neuroblastoma, Cancer Lett 228, 143-153). Therefore, SH-SY5Y cells are an ideal system to test cSTAR.

Experiments used a custom made RPPA with 115 validated antibodies listed in Table S1A below to interrogate the activities of signaling pathways involved in TrkA/B signaling (Schramm et al., 2005), including MAPK (RAS-ERK, JNK, p38), PI3 kinase (AKT, mTORC1/2), JAK-STAT, PLCγ-PKC, TGFβ-SMAD, Wnt, cyclic-AMP, cell cycle (CDK1, Cyclins, Rb, p53, p21^{WAF}), apoptosis (BAX, BCL2, BCLx, Caspase 3), transcription factor (MYC, NFκB, JUN), and tyrosine kinase (SRC, EGFR, PDGFR, IGFR) pathways. In addition to the 115 antibodies, three controls are also included, Mouse 1, 2 and 3.

TABLE S1A

| | | |
|---|---|---|
| Bad P Ser136 | beta-Catenin | Bcl-xl |
| Bad P Ser112 | beta-Catenin P Ser33, | c-Jun N-term |
| | Ser37, Thr41 | |
| Bak | cdc25A | Caspase 3 |
| CrkL P Tyr207 | PTEN | Caspase 3 cleaved |
| HSP27 (HSPB1) P | Tsc-2 (Tuberin) P | CDK1 (p34cdc2) |
| Ser78 | Thr1462 | P Tyr15 |
| JAK1 | Tsc-2 (Tuberin) | p53 |
| MEK1/2 P Ser217/221 | p70 S6 Kinase P Thr389 | EGFR P Tyr1086 |
| Met P Tyr1234 | PTEN P | GSK-3-beta P Ser9 |
| | Ser380, Thr382, Thr383 | |
| PI3 Kinase p110-alpha | SAPK/JNK (JNK2) | GSK-3-beta |
| PKA RII P Ser96 | p70 S6 Kinase P | IRS-1 P S636/639 |
| | Thr421, Ser424 | |
| PKC (pan) P | GSK-3-alpha/beta P | PLC-gamma1 |
| Ser660 (beta-2) | Ser21/Ser9 | |
| PKC substrate P | Stat6 P Tyr641 | Smad2 P Ser465, |
| (R/K)X(S*)(Hyd)(R/k) | | Ser467 |
| PKC-zeta | p53 P Ser15 | Smad3 P Ser423, |
| | | Ser425 |
| PKC-zeta/lambda P | p38 MAPK | CDK2 |
| Thr410/403 | PThr180, Tyr182 | |
| CDK1 (cdc2) | p38 MAPK | Stat3 |
| Met P Tyr1349 | mTOR P Ser2448 | Rsk2 Pser 227 |
| IGF-1R beta P | mTOR | NFkB p105/p50 |
| Tyr1162, Tyr1163 | | |
| ErbB-1/EGFR | Stat1 P Ser727 | PDGFR P Tyr1021 |
| ErbB-2/Her2/EGFR P | Raf P Ser259 | PDGFR P Tyr751 |
| Tyr1248/Tyr1173 | | |
| ErbB-3/Her3/EGFR | PLC-gamma1 P Tyr783 | Bcl-2 P Ser70 |
| ErbB-3/Her3/EGFR | PDK-1 P Ser241 | IGF-1R beta |
| P Tyr1289 | | |
| Stat5 | p90 S6 kinase (Rsk1-3) | Rock1 (C8F7) |
| | P Thr359, Ser363 | |
| Stat5 P Tyr694 | PDK-1 | B-Raf [EP152Y] |
| EGFR P Tyr1173 | p70 S6 Kinase | YAP1 [EP1674Y] |
| Akt P Thr308 | JAK1 P Tyr1022, Thr1023 | RhoA (67BC) |
| Met | c-Myc P Thr58, Ser62 | YAP P Ser127 |
| S6 Ribosomal | SAPK/JNK P Thr183, | PKC-alpha |
| protein P Ser235, | Tyr185 | |
| Ser236 | | |
| p44/42 MAPK | S6 Ribosomal protein p | GAPDH |
| (ERK1/2) | Ser240, Ser244 | |
| p44/42 MAPK | S6 Ribosomal Protein | Mouse_1 [control] |
| (ERK1/2) P | | |
| Thr202/Thr185, | | |
| Tyr204/Tyr187 | | |
| Src | Rb P Ser807, Ser811 | Mouse_2 [control] |
| Akt | Rb P Ser780 | Cyclin D1 |
| Akt P Ser473 | Raf P Ser338 | p21 CIP/WAF1 |
| beta-actin | MAPKAPK-2 P Thr334 | Stat3 P Tyr705 |
| NFkB p65 Ser536 | Bax | CrkL |
| cdc25c P Ser216 | Stat1 P Tyr701 | HSP27 (HSPB1) |
| c-Jun P Ser73 | Src (family) P Tyr416 | Ras |
| c-Myc | Smad2/3 P Ser465/Ser423, | Stat1 |
| | Ser467/Ser425 | |
| Rb | Smad1/5 P Ser463/Ser465 | Mouse_3 [control] |
| 4E-BP1 P Ser65 | Cyclin D1 P Thr286 | |
| 4E-BP1 P Thr37, | Bcl-2 | |
| Thr46 | | |

### List of Antibodies Used to Interrogate the Activities of Signaling Pathways

For each antibody, RPPA phosphoproteomics data was collected, measured as raw fluorescent intensity values in TrkA and TrkB cells stimulated with a ligand and treated with different inhibitors. A sample of the data is reproduced in Table S1B below, showing the measured fluorescent intensity values for a small sample of antibodies and a small sample of treatment and stimulation conditions. The full data set, including replicated experiments, contains 118 rows (one per antibody) and 144 columns (each containing 118 measurements and relating to an experimental set of treatment and stimulation conditions, which include replicated experiments).

TABLE S1B

| | TrkA | | TrkB | | TrkA |
|---|---|---|---|---|---|
| | cells S6K | | cells RSK | | cells MEK |
| | inhibitor | | inhibitor | | inhibitor |
| | 10 min NGF | | No GF | | 45 min NGF |
| Antibody | stimulation | . . . | stimulation | . . . | stimulation |
| Bad P Ser136 | 0.108983313 | . . . | 0.072253872 | . . . | 0.070242054 |
| Bad P Ser112 | 0.118124584 | . . . | 0.102985728 | . . . | 0.285306701 |
| Bak | 0.210965692 | . . . | 0.188778571 | . . . | 0.070903446 |
| CrkL P Tyr207 | 0.28135756 | . . . | 0.285229965 | . . . | 0.117434729 |
| HSP27 (HSPB1) | 0.293860125 | . . . | 0.322125782 | . . . | 0.043491502 |
| P Ser78 | | | | | |
| JAK1 | 0.100451464 | . . . | 0.106375337 | . . . | 0.051596339 |
| . . . | . . . | . . . | . . . | . . . | . . . |

### Representative Sample of RPPA Data Collected for a Subset of Antibodies and a Subset of Treatments/Stimulation Conditions

In addition, TrkA and TrkB activities were measured by Western blotting, as shown in Table S2 below. These antibodies detect phosphorylation sites that change protein activities or protein abundances. We measured pathway activities in untreated cells and cells treated with NGF (TrkA ligand) or BDNF (TrkB ligand) for 10 or 45 minutes. After normalization we calculated the fold changes in protein phosphorylation levels or abundances producing a data point for each protein.

TABLE S2

| Cell line_ drug | |
|---|---|
| treatment_timepoint of | |
| ligand stimulation_replicate | phospho-Trk |
| TrkA_DMSO_10_r1 | 2.182839665 |
| TrkA_DMSO_10_r2 | 4.547635969 |
| TrkA_DMSO_10_r3 | 5.101703397 |
| TrkA_DMSO_45_r1 | 4.116548071 |
| TrkA_DMSO_45_r2 | 2.568533055 |
| TrkA_TRKinh_10_r1 | 0.88708204 |
| TrkA_TRKinh_10_r3 | 0.606538683 |
| TrkA_TRKinh_45_r1 | 0.723971267 |
| TrkB_DMSO_10_r1 | 15.80103413 |
| TrkB_DMSO_10_r2 | 9.648417702 |
| TrkB_DMSO_10_r3 | 8.346156768 |
| TrkB_DMSO_45_r1 | 9.042776813 |
| TrkB_DMSO_45_r2 | 4.037015138 |
| TrkB_TRKinh_10_r1 | 3.715596867 |
| TrkB_TRKinh_10_r2 | 1.407950499 |
| TrkB_TRKinh_45_r1 | 3.539213927 |
| TrkA_DMSO_10_r6 | 5.101703397 |
| TrkA_DMSO_45_r4 | 4.116548071 |
| TrkB_DMSO_10_r4 | 15.80103413 |
| TrkB_DMSO_45_r4 | 9.042776813 |

### Measurement of Phospho-Trk Activities

In terms of the computational approach to processing the acquired data, each analyte level was first normalized on the GAPDH level, and then on the value of the same analyte in the absence of inhibitors and ligand stimulation to obtain fold-changes.

### Separating Distinct Physiological States and Building the State Transition Vector (STV)

The individual data points for TrkA and TrkB cells can be perceived as points in the molecular data space of 115 dimensions (corresponding to the measurement of 115 protein features) that describe the cell states. However, phenotypically SH-SY5Y cells exhibit only three different states: (1) a common ‘ground’ state of isogenic TrkA and TrkB cells with no GF stimulation, (2) a differentiation state following TrkA cell stimulation with NGF, and (3) a proliferation state following TrkB cell stimulation with BDNF. This suggests that not all data points are equally important in defining a cell state, and that distinct states might be determined by a handful of different patterns that are hidden in the molecular data.

Consequently, transitions between different cell states can be described by a few critical parameters, which for complex systems are termed the order parameters (Haken, H. (2004). Synergetics: Introduction and Advanced Topics (Springer); Landau, L. D., and Lifshitz, E. M. (1980). CHAPTER XIV—PHASE TRANSITIONS OF THE SECOND KIND AND CRITICAL PHENOMENA. In Statistical Physics (Third Edition), L. D. Landau, and E. M. Lifshitz, eds. (Oxford: Butterworth-Heinemann), pp. 446-516). While in physics the order parameters are found using theoretical models of state transitions, at present no mechanistic models can determine the dynamic changes in whole-cell signaling patterns between distinct cell states (Needham, E. J., Parker, B. L., Burykin, T., James, D. E., and Humphrey, S. J. (2019). Illuminating the dark phosphoproteome. Science Signaling 12, eaau8645).

To address this gap we developed the STV, which allows us to determine how signaling data patterns of a given cell state would have to change to enable the transition from one cell state into another.

The first step is to distinguish and separate distinct cell states in protein phosphorylation and/or expression molecular data space, using machine learning (ML) methods to cluster and classify signaling patterns. Two different unsupervised ML methods, Ward's hierarchical clustering and the K-means clustering (Duda, R. O., Hart, P. E., and Stork, D. G. (2012). Pattern Classification (Wiley)) generated identical results and determined two distinct sets of data points that correspond to two different cell states, NGF-stimulated TrkA differentiation state and BDNF-stimulated TrkB proliferation state.

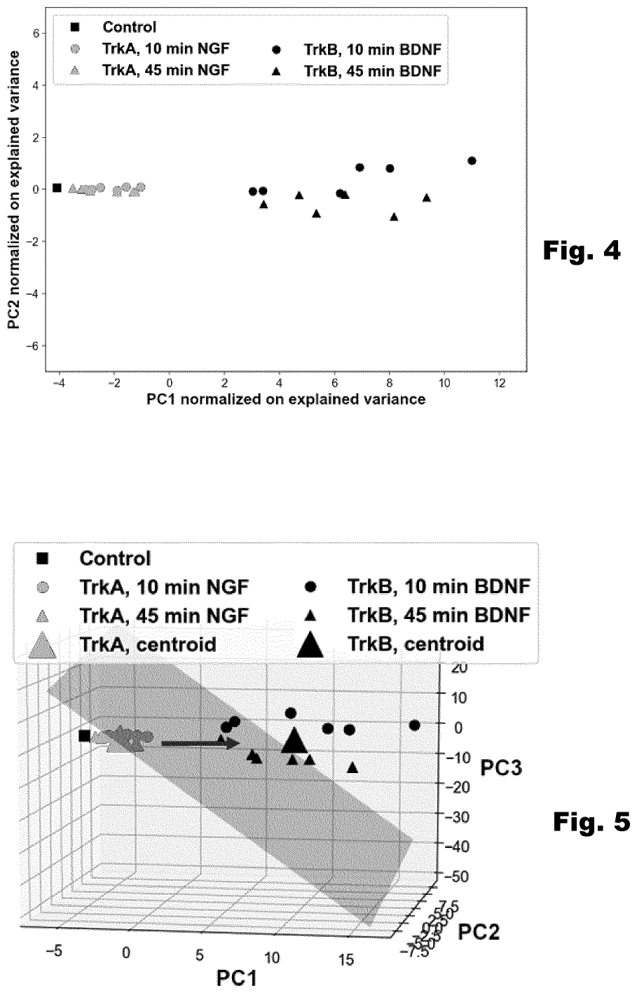

FIG. 4 shows the data points with a distinct separation between the TrkA differentiation and TrkB proliferation data points. In FIG. 4, PCA compressed RPPA data for TrkA and TrkB cells are plotted in the space of the first two principal components that are normalized by the data variance captured by these components. Following 100 ng/ml GF stimulation, the obtained data points in a 115-dimensional molecular dataspace were clustered using the K-means clustering (K=2). All data points from NGF-stimulated TrkA cells come out in a single cluster shown in in a lighter shade, left, and all data points from BDNF-stimulated TrkB cells appeared in a cluster shown in black, right. Control point without GF stimulation is shown as a black square.

Pandas Python library (Mckinney, W.a.o. (2010). Data structures for statistical computing in python. Paper presented at: Proceedings of the 9th Python in Science Conference (Austin, TX)) was used for RPPA data analysis and manipulation. For PCA compression and K-means data clustering we used the scikit-learn Python library (Pedregosa, F. et al., (2011). Scikit-learn: Machine Learning in Python. J Mach Learn Res 12, 2825-2830.).

R base functions (Team, R. C. (2013). R: A Language and Environment for Statistical Computing, R.F.f.S. Computing, ed. (Vienna, Austria)) and the pheatmap R package (Kolde, R. (2015). pheatmap: Pretty heatmaps [Software]. R. package) were used for Ward's hierarchical clustering and building heatmap.

Following stimulation with growth factors or drug perturbations, the fold changes in the phosphorylation levels or abundances of each protein are depicted in the molecular dataspace with the Cartesian coordinates. Then we applied a SVM, which is a supervised ML algorithm (Steinwart, I., and Christmann, A. (2008). Support Vector Machines (Springer Publishing Company, Incorporated)), to build a maximum margin hyperplane that maximizes the separation (aka margin) between distinct phenotypic states in the multidimensional dataspace of the RPPA data.

The SVM algorithm with a linear kernel from scikit-learn python library was applied to build a maximum margin hyperplane in the molecular dataspace that distinguish different cell states. The separation hyperplane is defined as,

$$( \vec{x} , \vec{n} ) = h . \qquad ( 1 )$$

Here \vec{x} is a radius vector from the origin of the coordinates to any point on the separation hyperplane, \vec{n} is the vector of unit length that is orthogonal to the separation hyperplane, and h is a constant.

To visualize the data and the state separation hyperplanes, we use principal component analysis (PCA). FIG. 5 shows the projections of both the data and the separation hyperplane into the first three PCA components that compress the multidimensional molecular dataspace. The TrkA differentiation point cloud is shown in a lighter shade, left and TrkB proliferation states are shown in black, right, with the separation hyperplane shown in grey along with the STV as a heavy arrow.

The second step is building a vector, which connects the centroids of the point clouds that represent the two phenotypic states. i.e. differentiation and proliferation. To determine the components contributing to this centroid-connecting vector, we calculate the difference of fold-changes in the detected phosphorylation levels or abundances between the centroids of the TrkA and TrkB point clouds for each protein. Dividing this centroid-connecting vector by its length, we define a state transition vector (STV); its projection to PCA space is shown as an arrow in FIG. 5. Thus, the STV is a vector of unit length, which determines the direction of the motion in the molecular dataspace that crosses the state separation surface and converts a given cell phenotypic state to a distinct state. For definiteness, in this description we will consider the STV direction from the centroid of the differentiation point cloud (TrkA cells) to the centroid of the proliferation point cloud (TrkB cells).

### Derivation of the STV

Let A be the centroid of a cloud of points Aᵢ (i=1, 2 . . . ) that corresponds to state 1 and B be the centroid of the point cloud Bᵢ, corresponding to state 2. A state transition vector (STV) from state 1 to state 2 is defined as a vector \vec{s} of unit length that has the same direction as the vector \vec{AB} connecting the centroids A and B,

$$\vec{s} = \vec{A B} / \vert \vec{A B} \vert . \qquad ( 2 )$$

Eq. 2 shows that the STV is initially built in the full molecular dataspace of 115 dimensions.

### Constituents of a Core Signaling Network.

Global cell signaling network spans multiple layers, from receptors to the cytoplasmic signaling layer and to the transcription factor layer (Citri, A., and Yarden, Y. (2006). EGF-ERBB signalling: towards the systems level. Nat Rev Mol Cell Biol 7, 505-516). Information transfer and interactions between these layers ultimately determine changes in cell state. Importantly, the STV allows us to capture the relative contributions of individual proteins to the overall change in molecular data that will switch TrkB proliferation state into TrkA cell differentiation state.

The vector s determined by Eq. 2 in the Cartesian coordinates has the components sₖ, k=1, . . . , N. Each STV component sₖ corresponds to an analyte k, measured by an antibody to a specific phosphosite on a protein or the protein abundance. The absolute value |sₖ| determines the STV rank of the analyte k, telling us about its importance for the switching of cell states. These STV ranks for a subset of the analytes with the highest rankings are presented in Table S3 (it being understood that the full table from which Table S3 is extracted would include all of the 115 analytes). To generate this table, the highest rank proteins and some of their immediate effectors were selected as core signaling network components. The changes in individual protein activities or abundances between the centroids of the data point clouds that characterize two different cells states were projected onto the STV to determine protein ranks. Resulting high rank proteins constitute a core signaling network.

TABLE S3

| | Contribution | |
|---|---|---|
| Analyte | to the STV | |
| pERBB1-2 | 0.493 | Core network |
| pTRK | 0.423 | components |
| AKT P Thr308 | 0.340 | |
| AKT P Ser473 | 0.332 | |
| ppERK | 0.286 | |
| pRPS6 | 0.265 | Other components of |
| S235, S236 | | global signalling |
| p-p38 | 0.213 | network |
| ppMEK | 0.202 | |
| pRPS6 | 0.112 | |
| S240, S244 | | |
| . . . | . . . | |

If the protein vector and the STV are parallel or antiparallel, the projection of the STV to the protein's axis in the multidimensional space equals the full length of the individual protein vector while the length of the projection decreases as the direction of the two vectors diverge, becoming zero when these vectors are orthogonal. Therefore, these STV projections capture the relative contributions of different individual proteins to the overall direction of change in protein activities or abundances that will convert cell fates.

Consequently, the STV allows us to directly assign ranks to individual proteins according to their importance in switching cell states based on the magnitude of their contributions to the STV. That means we can identify the components of a core signaling network that controls cellular responses, as identified in the rightmost column of Table S3.

We observe that proteins belonging to the peripheral layers of cell signaling have the highest ranks, i.e. (i) receptor tyrosine kinases (RTKs), which include TrkA, TrkB, EGFR and ERBB2 Volinsky, N., and Kholodenko, B. N. (2013). Complexity of receptor tyrosine kinase signal processing. Cold Spring Harb Perspect Biol 5, a009043); and (ii) soluble kinases, AKT, RAF, MEK and ERK. This may not surprise as receptors control many downstream signaling pathways, and the ERK and AKT pathways are considered main downstream effectors of TrkA/B receptor signaling (Vaishnavi, A., Le, A. T., and Doebele, R. C. (2015). TRKing Down an Old Oncogene in a New Era of Targeted Therapy. Cancer Discovery 5, 25).

Interestingly, other highly ranked effectors are p70S6K (S6K) and p90RSK (RSK) kinases, which are targets where ERK and AKT signaling converge (Abe et al., 2009). This indicates that the differential integration of ERK and AKT activities may be a key factor in determining different cell fates in this cell system.

In summary, the STV allows us to identify the signaling molecules that control cell fate decisions. The highest ranked molecules can be perceived as the components of a core signaling network that controls the larger network in terms of cell fate decisions.

Determining which components belong to the core signaling network can be treated as an optimization: determining a cut-off in the ranking which maximises the number of components which can be mapped onto existing biochemical pathways while minimising the total number of ranked components used.

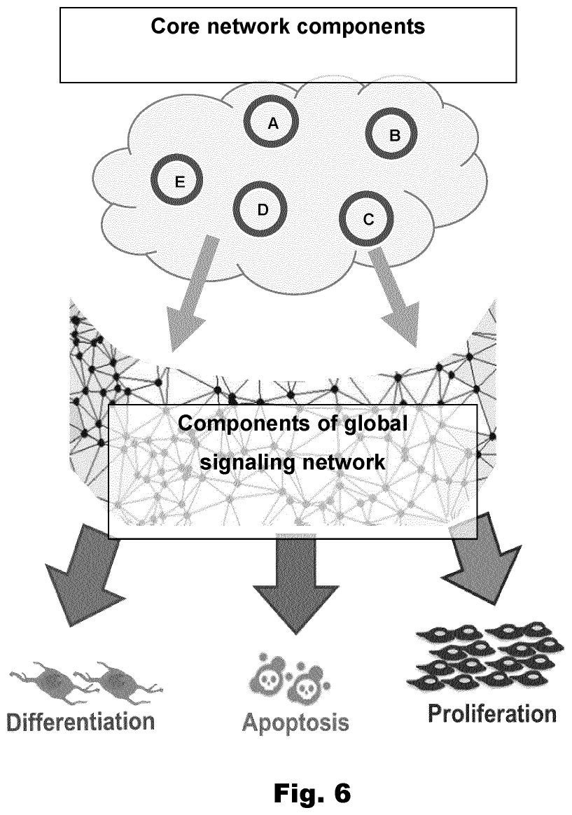

FIG. 6 shows a representation of the signaling network in terms of the core components identified from Table S3 and the remainder of the global signaling network as it affects differentiation, apoptosis and proliferation.

Next, we tested whether this knowledge can be directly used to design interventions that purposefully can change cell fates.

### Designing Perturbation Experiments Based on Linear Approximations of Biological Outcomes

The strategy considered is to experimentally perturb these core components and test whether these perturbations can change the cell states. In order to analyze and predict the effects of such perturbations we can take advantage of the fact that the STV also contains information about the contributions made by all the other components of the signaling network measured by the RPPA. Therefore, removing the core components from the STV slightly reduces the dimensionality of the STV but renders it a representation of the overall signaling network downstream of the core components. It also eliminates potentially confounding effects resulting from the perturbations indirectly affecting the activity of upstream network components through feedback loops.

For instance, inhibition of ERK will abolish negative feedbacks to TrkA/B mediated RAS activation (Lake, D. et al. (2016). Negative feedback regulation of the ERK1/2 MAPK pathway, Cell Mol Life Sci 73, 4397-4413; Lavoie, H. et al. (2020). ERK signalling: a master regulator of cell behaviour, life and fate, Nature Reviews Molecular Cell Biology 21, 607-632), which would register as a change in ERK signaling, however is inconsequential for ERK mediated downstream events, as ERK is blocked by the inhibitor.

Thus, we use this dimensionality reduced STV for estimating the network effects and biological outcomes of experimental perturbations. For each perturbation we determine a perturbation vector that connects the centroids of the point clouds that have been obtained before and after the perturbation. This vector changes the cell phenotype when it pushes the centroid of the original point cloud across the plane that separates different cell states, stabilizes a cell state when moving the centroid away from the separation plane, or has no effect when it is (nearly) parallel to the separation plane.

These outcomes can be quantified by determining the dot product (P) of the perturbation vector and the dimensionality reduced STV. As the STV indicates the direction from a proliferation to a differentiation state, a negative P moves the proliferation state towards differentiation, which will be achieved when the value is large enough to cross the separation plane. Conversely, a positive P moves stabilizes the proliferation state, and P=0 does not change cell states.

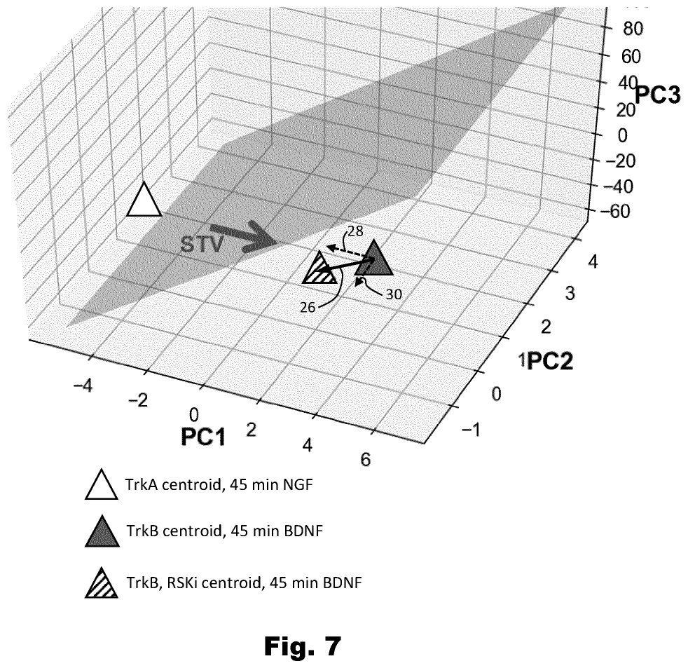

FIG. 7 shows the decomposition of a perturbation vector 26 into a vector 28 that is collinear with the STV and a vector 30 that is perpendicular to the STV. TrkB cells were treated with the p90RSK inhibitor BI-D1870. Data points were acquired corresponding to 45 min 100 ng/ml GF stimulation, and these were projected into the first 3 principal components in order to calculate the point cloud centroids. For clarity, only the centroids are shown in FIG. 7, corresponding to: TrkA 45 minute NGF, TrkB 45 minute BDNF before perturbation, and the perturbed centroid TrkB RSKi 45 minute BDNF. It can be seen that the component 28 moves the proliferation state towards differentiation, while the perpendicular component 30 has no effect in this regard.

### Derivation of a Projection of a Perturbation Vector on the STV

For a correct interpretation of perturbation data using the STV, we have to exclude the analytes that composed the modules of our core signaling network. Accordingly, the dimensionality of the molecular dataspace where the STV and perturbation vectors are calculated is reduced from 115 to 70, due to 45 analytes from Table S1A, related to the five proteins flagged in Table S3, being allocated to core network components, leaving the remaining 70 analytes to defined the reduced dimensionality dataspace.

Let A with the radius-vector \vec{x}_{A} be the centroid of the point cloud Aᵢ, corresponding to the unperturbed state 1. Let Aₚₑᵣₜ with the radius-vector \vec{x}_{A}_{ₚₑᵣₜ} be the centroid of the point cloud (Aᵢ^{pert}), corresponding to the perturbed state 1. Then the perturbation vector is defined as,

$${\vec{x}}_{{A}_{pert}} - {\vec{x}}_{A} = \vec{A {A}_{pert}} = \vec{P} . \qquad ( 3 )$$

The projection P of the perturbation vector \vec{P} on the STV (\vec{s}) is obtained as a dot product of these two vectors,

$$P = ( \vec{P} , \vec{s} ) . \qquad ( 4 )$$

Distance from a Data Point to the Separation Plane Along the STV

Starting from a data point A in the molecular dataspace, we build a vector, which is collinear with the STV (\vec{s}) and crosses the separation hyperplane at a point, Aₛ. Thus, we have,

$$\vec{A {A}_{s}} = S \cdot \vec{s} . \qquad ( 5 )$$

If the vector \vec{AA_{S}} has the same direction as the STV, the value of S is positive, and S is negative if the vector \vec{AA_{S}} has the opposite direction to the STV. In either scenario, the length (|S|) of the vector \vec{AA_{S}} is the distance from the point A to the separation surface along the STV.

The vectors \vec{x}_{A}_{_{S}} and \vec{x}_{A} connecting the origin of the coordinates and the points A_{S} and A, respectively, are related by the following equation,

$${\vec{x}}_{{A}_{s}} = {\vec{x}}_{A} + S \cdot \vec{s} . \qquad ( 6 )$$

Using Eqs. 1, 5 and 6, we obtain,

$$( {\vec{x}}_{{A}_{s}} , \vec{n} ) = ( ( {\vec{x}}_{A} + S \cdot \vec{s} ) , \vec{n} ) = ( {\vec{x}}_{A} , \vec{n} ) + S \cdot ( \vec{s} , \vec{n} ) = h . \qquad ( 7 )$$

Eq. 7 allows us to calculate the distance |S| from a point A in the molecular dataspace to the separation hyperplane between two different cell states, as follows

$$\vert S \vert = \vert ( h - ( {\vec{x}}_{A} , \vec{n} ) ) / ( \vec{s} , \vec{n} ) \vert . \qquad ( 8 )$$

If \vec{s}=\vec{n} then |S| is the shortest distance to the separation hyperplane. If \vec{s}≠\vec{n} then |S| is larger than the shortest distance to the hyperplane, because vectors \vec{s} and \vec{n} have unit lengths. If point A is a centroid of a point cloud that corresponds to a distinguishable cell state, than Eq. 8 determines the distance of this centroid to the separation hyperplane.

For experimental testing we used small molecule inhibitors to target core components:
As there are no highly TrkA/B selective inhibitors, we used SP600125, which inhibits both TrkA and TrkB (Jung, E. J., and Kim, D. R. (2010). Control of TrkA-induced cell death by JNK activation and differential expression of TrkA upon DNA damage. Molecules and Cells 30, 121-125).
Because SP600125 also inhibits the cJun N-terminal Kinase (JNK) (Bennett, B. L. et al. (2001). SP600125, an anthrapyrazolone inhibitor of Jun N-terminal kinase. Proceedings of the National Academy of Sciences 98, 13681), we used JNK-IN-8, which inhibits JNK but not the Trk receptors (Zhang, T. et al., (2012). Discovery of potent and selective covalent inhibitors of JNK. Chem Biol 19, 140-154), to dissect the impact of the JNK inhibition.
AKT was blocked by the AKT inhibitor IV.
To inhibit p70S6 kinase, which phosphorylates the ribosomal RPS6 protein), we used LY2584702.
To perturb the ERK pathway, we used the MEK inhibitor Trametinib, which inhibits the kinase that activates ERK, and BI-D1870, which inhibits p90RSK, a kinase downstream of ERK.

Generally, the predicted effects of the inhibitors on the signaling network correlated well with the biological outcomes (Table 1 below). As predicted, the large negative P values for TrkB or p70S6K inhibition also strongly increased differentiation. Interestingly, and also as predicted, the RSK inhibitor had differential effects, decreasing differentiation in TrkA cells and weakly increasing differentiation in TrkB cells.

TABLE 1
Dot products of the STV and perturbation vectors and biological outcomes.

| | SY5Y-TrkA ± NGF | | SY5Y-TrkB ± BDNF | |
|---|---|---|---|---|
| | Dot | | Dot | |
| | products of the | | products of the | |
| | dimensionality | | dimensionality | |
| | reduced STV and | | reduced STV and | |
| | the perturbation | | the perturbation | |
| Perturbation | vectors (P) | Experiment | vectors (P) | Experiment |
| Trk inhibitor | −2.608601 | Increased | −9.979079 | Strong increase in |
| | | differentiation | | differentiation |
| MEK inhibitor | −1.263213 | No effect | −0.628089 | Weak increase in |
| | | | | differentiation |
| AKT inhibitor | −2.211122 | Increased | −2.241621 | Increase in |
| | | differentiation | | differen- |
| | | | | tiation |
| p70S6K inhibitor | −1.867636 | Increased | −8.190953 | Strong increase in |
| | | differentiation | | differentiation |
| p90RSK inhibitor | 2.65654 | Decreased | −1.394221 | Weak |
| | | differentiation | | increase in |
| | | | | differentiation |
| JNK inhibitor | −0.694788 | No effect | −0.155327 | No effect |

Negative P values indicate a shift from proliferation to differentiation.

These correlations show that the STV produces good predictions which perturbations can change cell states. In Waddington's terms, the STV predicts well how we can steer a marble into a valley but does not reveal why that steer works. In order to obtain mechanistic insights into how a cell ‘computes’ its fate decisions after experimental perturbations, we need to reconstruct the computing machinery, i.e., the connections of a core network, and establish how perturbations to its constituents affect the DPD. The only means to precisely predict and explain the outcome of these experimental manipulations, which maneuver a cell through a Waddington landscape, is to explicitly model the nonlinear signaling dynamics that determine cell state transitions.

Explaining STV Predictions and Cell State Transitions by Mechanistic Insights Gained from Non-Linear Dynamic Models

In this context, an informative mechanistic model needs to comprise (i) a faithfully reconstructed network topology of the core network components deduced from the STV with interaction signs and strengths; and (ii) a network node that summarizes the remainder of the global network controlled by the core network and which links signaling changes to phenotypical changes; we call this node the dynamic phenotype descriptor (DPD).

### Calculation of the DPD Module Output (S) Using Experimental Data

In the molecular dataspace, we consider the STV as a vector \vec{s} of unit length directed from a centroid of a differentiation TrkA point cloud to a centroid of a proliferation TrkB point cloud. We now define the output of the DPD module as the S value,

$$DPD = S = ( h - ( {\vec{x}}_{A} , \vec{n} ) ) / ( \vec{s} , \vec{n} ) . \qquad ( 9 )$$

In this definition, the direction of the vector \vec{n}, which is orthogonal to the separation hyperplane, points from the TrkB cloud to the TrkA cloud. Then, the DPD value (S) is positive for proliferation TrkB points and negative for differentiation TrkA points. The DPD values for ground state of TrkA and TrkB cells, GF stimulations and inhibitor treatments are given in Tables S5A and S5B.

TABLE S5A
BMRA-reconstructed incidence (A) matrices showing mean and STD values for matrix elements
Matrix A, mean ± std

| TrkA, | | | | | | | |
|---|---|---|---|---|---|---|---|
| 10 min | TRK | ERK | AKT | JNK | S6K | RSK | ERBB |
| TRK | −1 | 0 | 0 | 0 | 0 | 0 | 0 |
| ERK | 1 ± 0 | −1 | 1 ± 0 | 1 ± 0 | 1 ± 0 | 0 | 0.89 ± 0.32 |
| AKT | 1 ± 0 | 1 ± 0 | −1 | 0 | 1 ± 0 | 1 ± 0 | 0 |
| JNK | 0 | 0 | 0 | −1 | 0 | 0 | 0 |
| S6K | 0 | 0 | 1 ± 0 | 0 | −1 | 0 | 0 |
| RSK | 0 | 0 | 0 | 0 | 0 | −1 | 0 |
| ERBB | 0 | 0 | 0 | 0 | 1 ± 0 | 0 | −1 |

| TrkA, | | | | | | | | |
|---|---|---|---|---|---|---|---|---|
| 45 min | TRK | ERK | AKT | JNK | S6K | RSK | ERBB | DPD |
| TRK | −1 | 0 | 0 | 0 | 0 | 0 | 0 | 0 |
| ERK | 1 ± 0 | −1 | 1 ± 0 | 0 ± 0.05 | 1 ± 0 | 0.17 ± 0.37 | 1 ± 0 | 0 |
| AKT | 1 ± 0 | 1 ± 0 | −1 | 1 ± 0 | 1 ± 0 | 1 ± 0 | 1 ± 0 | 0 |
| JNK | 0 | 0 | 0 | −1 | 0 | 0 | 0 | 0 |
| S6K | 0 | 0 | 1 ± 0 | 0 | −1 | 0 | 0 | 0 |
| RSK | 0 | 1 ± 0 | 0 | 0 | 0 | −1 | 0 | 0 |
| ERBB | 1 ± 0 | 1 ± 0 | 0 | 0 | 1 ± 0 | 0 | −1 | 0 |
| DPD | 0 | 1 ± 0 | 0 | 1 ± 0 | 1 ± 0 | 1 ± 0 | 0 | −1 |

| TrkB, | | | | | | | |
|---|---|---|---|---|---|---|---|
| 10 min | TRK | ERK | AKT | JNK | S6K | RSK | ERBB |
| TRK | −1 | 0 | 0 | 0 | 0 | 0 | 0 |
| ERK | 0.78 ± 0.41 | −1 | 0.05 ± 0.21 | 0.77 ± 0.42 | 0 | 0.85 ± 0.36 | 1 ± 0 |
| AKT | 1 ± 0 | 1 ± 0 | −1 | 1 ± 0 | 1 ± 0 | 1 ± 0 | 1 ± 0 |
| JNK | 0 | 1 ± 0 | 0 | −1 | 0 | 0 | 0.67 ± 0.47 |
| S6K | 0 | 0 | 1 ± 0 | 0 | −1 | 0 | 0 |
| RSK | 0 | 1 ± 0 | 0 | 0 | 0 | −1 | 0 |
| ERBB | 1 ± 0 | 1 ± 0 | 0 | 0 | 0 | 1 ± 0 | −1 |

| TrkB, | | | | | | | | |
|---|---|---|---|---|---|---|---|---|
| 45 min | TRK | ERK | AKT | JNK | S6K | RSK | ERBB | DPD |
| TRK | −1 | 0 | 0 | 0 | 0 | 0 | 0 | 0 |
| ERK | 1 ± 0 | −1 | 0 | 0.07 ± 0.25 | 0 | 0.47 ± 0.5 | 0.47 ± 0.5 | 0 |
| AKT | 0.01 ± 0.11 | 1 ± 0 | −1 | 0 | 0.57 ± 0.49 | 0.61 ± 0.49 | 1 ± 0 | 0 |
| JNK | 0 | 1 ± 0 | 0 | −1 | 0 | 0 | 0.62 ± 0.49 | 0 |
| S6K | 0 | 1 ± 0 | 1 ± 0 | 0 | −1 | 0 | 0 | 0 |
| RSK | 0 | 1 ± 0 | 0 | 0 | 0 | −1 | 0 | 0 |
| ERBB | 1 ± 0 | 0 | 1 ± 0 | 0 | 0 | 1 ± 0 | −1 | 0 |
| DPD | 0 | 1 ± 0 | 0 | 0 | 1 ± 0 | 0 | 0 | −1 |

TABLE S5B
BMRA-reconstructed connection (r) matrices showing mean and STD values for matrix elements
Matrix r, mean ± std

| TrkA, | | | | | | | |
|---|---|---|---|---|---|---|---|
| 10 min | TRK | ERK | AKT | JNK | S6K | RSK | ERBB |
| TRK | −1 | 0 | 0 | 0 | 0 | 0 | 0 |
| ERK | 1.23 ± 0.55 | −1 | −0.49 ± 0.55 | 0.95 ± 0.55 | −0.46 ± 0.54 | 0 | 0.42 ± 0.54 |
| AKT | −0.51 ± 0.73 | 1.02 ± 0.73 | −1 | 0 | −0.24 ± 0.73 | 0.61 ± 0.73 | 0 |
| JNK | 0 | 0 | 0 | −1 | 0 | 0 | 0 |
| S6K | 0 | 0 | 0.53 ± 0.03 | 0 | −1 | 0 | 0 |
| RSK | 0 | 0 | 0 | 0 | 0 | −1 | 0 |
| ERBB | 0 | 0 | 0 | 0 | 0.28 ± 0.18 | 0 | −1 |

| TrkA, | | | | | | | | |
|---|---|---|---|---|---|---|---|---|
| 45 min | TRK | ERK | AKT | JNK | S6K | RSK | ERBB | DPD |
| TRK | −1 | 0 | 0 | 0 | 0 | 0 | 0 | 0 |
| ERK | 0.21 ± 0.62 | −1 | 0.24 ± 0.62 | 0 ± 0.01 | −0.66 ± 0.62 | −0.03 ± 0.16 | 1.7 ± 0.62 | 0 |
| AKT | −0.61 ± 0.5 | 1.07 ± 0.5 | −1 | −0.83 ± 0.49 | −0.41 ± 0.49 | 1.64 ± 0.49 | 0.18 ± 0.49 | 0 |
| JNK | 0 | 0 | 0 | −1 | 0 | 0 | 0 | 0 |
| S6K | 0 | 0 | 0.53 ± 0.03 | 0 | −1 | 0 | 0 | 0 |
| RSK | 0 | 0.25 ± 0.03 | 0 | 0 | 0 | −1 | 0 | 0 |
| ERBB | −0.2 ± 0.52 | 0.43 ± 0.52 | 0 | 0 | 0.4 ± 0.52 | 0 | −1 | 0 |
| DPD | 0 | 1 ± 0.43 | 0 | −1.09 ± 0.43 | 1.05 ± 0.43 | −0.83 ± 0.43 | 0 | −1 |

| TrkB, | | | | | | | |
|---|---|---|---|---|---|---|---|
| 10 min | TRK | ERK | AKT | JNK | S6K | RSK | ERBB |
| TRK | −1 | 0 | 0 | 0 | 0 | 0 | 0 |
| ERK | 0.01 ± 0.61 | −1 | 0 ± 0.06 | 0.34 ± 0.61 | 0 | −0.08 ± 0.62 | 0.33 ± 0.66 |
| AKT | 0.24 ± 0.52 | 0.55 ± 0.52 | −1 | −0.07 ± 0.52 | −0.18 ± 0.52 | −0.06 ± 0.52 | 0.54 ± 0.52 |
| JNK | 0 | 0.87 ± 0.28 | 0 | −1 | 0 | 0 | −0.02 ± 0.28 |
| S6K | 0 | 0 | 0.49 ± 0.03 | 0 | −1 | 0 | 0 |
| RSK | 0 | 0.75 ± 0.02 | 0 | 0 | 0 | −1 | 0 |
| ERBB | 0.35 ± 0.54 | 0.61 ± 0.54 | 0 | 0 | 0 | 0.79 ± 0.54 | −1 |

| TrkB, | | | | | | | | |
|---|---|---|---|---|---|---|---|---|
| 45 min | TRK | ERK | AKT | JNK | S6K | RSK | ERBB | DPD |
| TRK | −1 | 0 | 0 | 0 | 0 | 0 | 0 | 0 |
| ERK | 1.03 ± 0.42 | −1 | 0 | 0.01 ± 0.11 | 0 | 0.26 ± 0.48 | −0.14 ± 0.42 | 0 |
| AKT | 0 ± 0.03 | 0.71 ± 0.64 | −1 | 0 | −0.08 ± 0.56 | −0.33 ± 0.62 | 0.72 ± 0.65 | 0 |
| JNK | 0 | 0.85 ± 0.28 | 0 | −1 | 0 | 0 | −0.16 ± 0.26 | 0 |
| S6K | 0 | 0.36 ± 0.16 | 0.35 ± 0.16 | 0 | −1 | 0 | 0 | 0 |
| RSK | 0 | 0.53 ± 0.02 | 0 | 0 | 0 | −1 | 0 | 0 |
| ERBB | 0.31 ± 0.6 | 0 | 0.23 ± 0.61 | 0 | 0 | 1.02 ± 0.61 | −1 | 0 |
| DPD | 0 | 0.79 ± 0.39 | 0 | 0 | 0.94 ± 0.39 | 0 | 0 | −1 |

Table S5C below shows the DPD module outputs panel, indicating the analytes which were taken as outputs of core signaling network modules.

TABLE S5C
Core signaling network output analytes

| | | Measurement |
|---|---|---|
| Module | Output analyte | technique |
| TRK | pTrk | Western Blot |
| ERBB | ErbB-2/Her2/EGFR P Tyr1248/Tyr1173 | RPPA |
| ERK | p44/42 MAPK (ERK1/2) P Thr202/Thr185, | RPPA |
| | Tyr204/Tyr187 | |
| AKT | Akt P Ser473 | RPPA |
| JNK | SAPK/JNK P Thr183, Tyr185 | RPPA |
| RSK | Rsk2 Pser 227 | RPPA |
| S6K | p70 S6 Kinase P Thr389 | RPPA |

### Calculations of the DPD Changes Upon Perturbations.

Using the STV (\vec{s}), a perturbation vector (\vec{P}) and the unit length vector (\vec{n}) orthogonal to the separation hyperplane, we can calculate how the DPD value changes following each inhibitor perturbation. The DPD values, determined for the centroids of unperturbed (A) and perturbed (Aₚₑᵣₜ) states, S and Sₚₑᵣₜ, respectively, are the following (see Eq. 9),

$$S = ( h - ( {\vec{x}}_{A} , \vec{n} ) ) / ( \vec{s} , \vec{n} ) . \qquad ( 10 )$$

$${S}_{pert} = ( h - ( {\vec{x}}_{{A}_{pert}} , \vec{n} ) ) / ( \vec{s} , \vec{n} ) . \qquad ( 11 )$$

Using Eqs. 3, 10 and 11, the change in the DPD upon a perturbation is expressed as follows,

$$Δ S = {S}_{pert} - S = ( {\vec{x}}_{A} - {\vec{x}}_{{A}_{pert}} , \vec{n} ) / ( \vec{s} , \vec{n} ) = - ( \vec{P} , \vec{n} ) / ( \vec{s} , \vec{n} ) . \qquad ( 12 )$$

From Eq. 11 it follows that if \vec{s}=\vec{n}, then ΔS_{c}=−P.

### Using Bayesian Modular Response Analysis (BMRA) for Reconstructing a Mechanistic Core Network.

We build upon a physics-based method, termed Modular Response Analysis (MRA) that can exactly reconstruct and quantify causal, local connections between network nodes, including feedback loops (Bastiaens, P. et al. (2015). Silence on the relevant literature and errors in implementation. Nat Biotechnol 33, 336-339; de la Fuente A. et al. (2002). Linking the genes: inferring quantitative gene networks from microarray data. Trends Genet 18, 395-398, 2002; Kholodenko et al., (2002). Untangling the wires: A strategy to trace functional interactions in signaling and gene networks. Proceedings of the National Academy of Sciences 99, 12841; Yalamanchili et al., (2006) Quantifying gene network connectivity in silico: scalability and accuracy of a modular approach. Syst Biol (Stevenage) 153, 236-246).

In the MRA framework, each node is a reaction module, which can be a single protein or gene, a signaling pathway, or any functional object that can be defined in terms of input-output relations. For instance, in our core network the ERK module is a three-tier pathway that includes all isoforms of RAF, MEK and ERK. The network topology is quantified in terms of connection coefficients, aka local responses or connection strengths (Kholodenko et al., (1997) Quantification of information transfer via cellular signal transduction pathways [published erratum appears in FEBS Lett 1997 Dec. 8; 419(1):150]. FEBS Lett 414, 430-434).

These cannot be directly measured, because responses propagate through a network masking direct connections. Only systems-level responses to perturbations are captured in experimental data, and MRA infers a network from these responses. The responses are measured when a network approaches a steady state, or at the time instances when a signaling response is near its maximum or minimum, because in both cases the time derivative is about zero (Kholodenko, B. N., and Kholodov, L. E. (1980). Individualization and optimization of dosings of pharmacological preparations; principle of maximum in the analysis of pharmacological response. Pharmaceutical Chemistry Journal 14, 287-291; Santos et al., (2007). Growth factor-induced MAPK network topology shapes Erk response determining PC-12 cell fate. Nat Cell Biol 9, 324-330; Sontag et al., (2004). Inferring dynamic architecture of cellular networks using time series of gene expression, protein and metabolite data. Bioinformatics 20, 1877-1886). Whereas the overall topology usually does not markedly change between early peak and steady state responses, the connection strengths are highly dynamic and necessarily change at different time moments after perturbation.

The original MRA method requires as many perturbations as there are nodes in a network, and it is sensitive to measurement noise in the data (Thomaseth et al., (2018). Impact of measurement noise, experimental design, and estimation methods on Modular Response Analysis based network reconstruction. Sci Rep 8, 16217).

To increase its robustness, statistical reformulations of MRA have been developed based on maximum likelihood and Bayesian algorithms (Klinger et al., (2013). Network quantification of EGFR signaling unveils potential for targeted combination therapy. Mol Syst Biol 9, 673; Santra et al., (2013). Integrating Bayesian variable selection with Modular Response Analysis to infer biochemical network topology. BMC Syst Biol 7, 57).

The Bayesian MRA formulation (BMRA) requires fewer perturbations than MRA, is tolerant to noise, and allows to incorporate existing pathway knowledge as a prior network to improve inference precision (Santra et al., (2018). Reconstructing static and dynamic models of signaling pathways using Modular Response Analysis. Current Opinion in Systems Biology 9, 11-21). Even when this knowledge is inaccurate for half of the network edges, BMRA recovers a nearly perfect network topology as validated in independent experiments (Halasz et al., (2016). Integrating network reconstruction with mechanistic modeling to predict cancer therapies. Sci Signal 9, ra114).

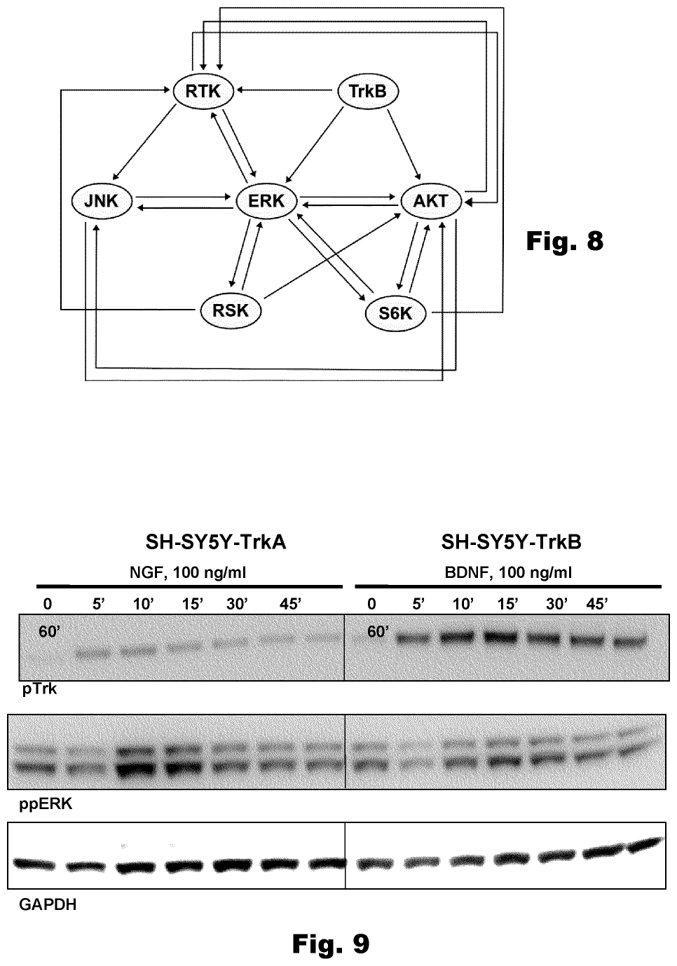

Mapping the core components specified by the STV onto known signaling pathways we obtained a prior topology of a core network, which contains the Trk and ERBB receptors, and downstream signaling pathways, i.e. the ERK, AKT, p70S6K, p90RSK and JNK pathways. This prior network was identical for the TrkA and TrkB expressing cells, as seen in FIG. 8, which shows the prior topology of core network connections based on the existing knowledge.

In order to reconstruct the posterior network, we used the drug perturbations described in Table 1, measuring 10 and 45 minute timepoints in TrkA and TrkB cells stimulated with NGF or BDNF, respectively. The time courses after growth factor stimulation indicated that the TrkA, TrkB, EGFR, ERBB2, AKT and ERK peaked around 10 minutes and attained steady-state levels at about 45 minutes, as seen in FIG. 9 which shows the time courses for pTRK and ppERK activation in TrkA and TrkB cells after stimulation with 100 ng/ml NGF or BDNF, respectively, measured by Western Blot.

Network reconstruction was performed for both time points using BMRA as shown in Table S4 below, and as described in the mathematical treatment that follows.

TABLE S4
DPD output values (S) calculated from RPPA data for different ligands
and drug perturbations at 45 minutes and experimentally measured
percentages of differentiated TrkA and TrkB cells at 72 hours.

| | | | | |
|---|---|---|---|---|
| | | | Experimentally | |
| | | Change in the | measured percentage | |
| | | DPD output | of differentiated | |
| Inhibitor | DPD output S | ΔS | cells | Conclusion |

Cell line SH-SY5Y-TrkA; Ligand NGF

| | | | | |
|---|---|---|---|---|
| No | −2.3 ± 0.3 | N/A | 57% ± 5% | N/A |
| TRK | −4.84 ± 0.16 | −2.53 ± 0.35 | 64.4% ± 2.5% | A |
| MEK | −3.0 ± 0.3 | −0.7 ± 0.4 | 48% ± 3% | A |
| AKT | −3.6 ± 0.5 | −1.3 ± 0.6 | 62% ± 3% | A |
| S6K | −3.8 ± 0.3 | −1.5 ± 0.4 | 41% ± 5% | A |
| RSK | −0.54 ± 0.13 | 1.8 ± 0.3 | 37.0% ± 2.4% | B |
| JNK | −2.4 ± 0.7 | −0.07 ± 0.75 | 45% ± 5% | A |

Cell line SH-SY5Y-TrkB; Ligand BDNF

| | | | | |
|---|---|---|---|---|
| No | 5.7 ± 0.6 | N/A | 17% ± 1% | N/A |
| TRK | −3.84 ± 0.03 | −9.6 ± 0.6 | 55% ± 4% | D |
| MEK | 6.1 ± 0.4 | 0.35 ± 0.72 | 20% ± 1% | A |
| AKT | 4.3 ± 0.8 | −1.5 ± 1.0 | 34.3% ± 0.8% | C |
| S6K | −2.10 ± 0.05 | −7.8 ± 0.6 | 45% ± 3% | D |
| RSK | 4.0 ± 0.2 | −1.7 ± 0.6 | 29% ± 4% | C |
| JNK | 5.95 ± 0.56 | 0.23 ± 0.82 | 25% ± 4% | A |

Key to conclusions column
A No statistically significant effects
B Decrease in differentiation
C Increase in differentiation
D Strong increase in differentiation

Although not surprisingly, connection strengths were different between the peak and steady-state levels, a common consensus network can readily be derived for each cell line.

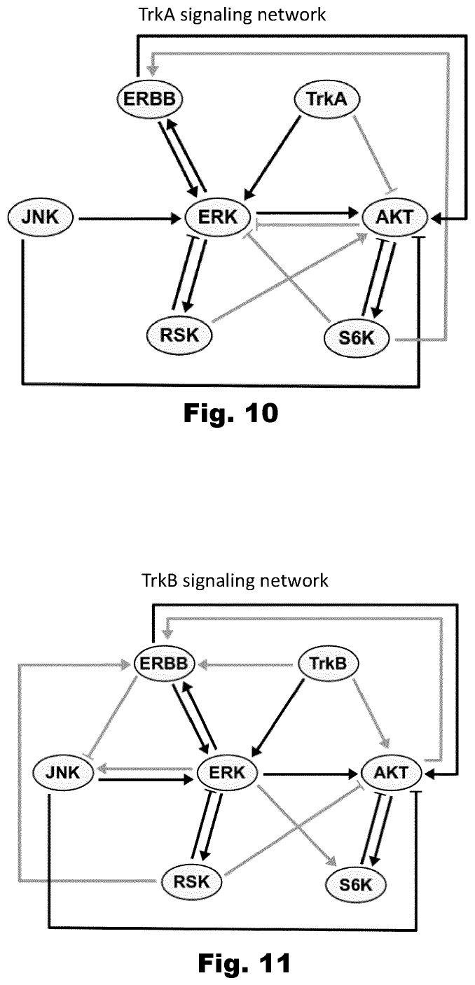

FIGS. 10 and 11 show how the inferred core signaling networks reconstructed by BMRA. The inferred topology of the TrkA core signaling network is shown in FIG. 10 and that of the TrkB core signaling network is shown in FIG. 11. Edges that are specific to TrkA and TrkB are shown in lighter colours in each of FIGS. 10 and 11 with the common edges shown in black. Arrowheads indicate activation, blunt ends indicate inhibition.

The BMRA-reconstructed TrkA and TrkB signaling networks feature numerous differences in their topologies. Major differences include a strong negative feedback from JNK to AKT in the TrkA network and a strong positive feedback loop from RSK to ERBB in the TrkB network that may act as an autocatalytic amplifier of the ERBB->ERK->RSK->ERBB module. The strong activation of p70S6K by ERK in TrkB cells is subverted into a strong inhibition of ERK by p70S6K in TrkA cells. Overall, the TrkA network has more inhibitory connections, while the TrkB network comprises more stimulatory interactions.

### Mathematical Basis of Bayesian Modular Response Analysis (BMRA) Network Inference

To reconstruct the topology and strengths of causal connections of the core network, including the influence of each pathway module on the Dynamic Phenotype Descriptor (DPD), we used a modified version of BMRA (Halasz et al., 2016). A family of Modular Response Analysis (MRA) methods, including BMRA, allows both (i) predicting systems-level network responses to different perturbations and (ii) reconstructing the topology and strengths of causal network connections based on experimentally measured responses to perturbations (Bastiaens et al., 2015; Santra et al., 2018).

Each core network module has a single quantitative output (xᵢ), termed communicating species in the MRA family framework. The temporal dynamics of the module outputs is given by a system of ordinary differential equations (ODE),

$$\frac{d {x}_{i}}{dt} = {f}_{i} ( {x}_{1} , … , {x}_{n} , p ) , i = 1 , … , n . \qquad ( 13 )$$

Here the functions ƒᵢ describe how the rate of change of independent variables xᵢ depends on the activities of other network modules. The parameters, pᵢ∈P, represent kinetic constants and any external or internal conditions, such as the conserved moieties and external concentrations that are maintained constants.

For each network module xᵢ, the connection coefficient (rᵢⱼ) quantifies the fractional change (Δxᵢ/xᵢ) in its output brought about by a change in the output of another module (Δxⱼ/xⱼ), while keeping the remaining nodes (xₖ, k≠i,j) unchanged to prevent the spread of this perturbation over the network (Kholodenko et al., 1997; Kholodenko et al., 2002).

$${r}_{ij} = \partial log {x}_{i} / \partial log {x}_{j} ; {x}_{k} = const ( k \neq i , j ) . \qquad ( 14 )$$

Positive and negative rᵢⱼ quantify direct activation and inhibition, respectively, whereas zero values show that there are no direct connections. The coefficients rᵢⱼ are expressed in terms of the elements of the Jacobian matrix (∂ƒᵢ/∂xⱼ) of the ODE system at the steady state (st. st.), as follows (Kholodenko et al., 2002),

$${{r}_{ij} = \frac{\partial log {x}_{i}}{\partial log {x}_{j}} = \frac{\partial {x}_{i}}{\partial {x}_{j}} ( \frac{{x}_{j}}{{x}_{i}} ) = - \frac{\frac{\partial {f}_{i} ( {x}_{1} , … , {x}_{n} , p )}{\partial {x}_{j}}}{\frac{\partial {f}_{i} ( {x}_{1} , … , {x}_{n} , p )}{\partial {x}_{i}}} ( \frac{{x}_{j}}{{x}_{i}} ) \vert}_{st . st .} i , j = 1 , … n . \qquad ( 15 )$$

The connection coefficients cannot be directly measured and are inferred using the systems-level, global network responses to perturbations. Following a change (Δpⱼ) in a parameter (pⱼ) that affects node j, the global response (Rᵢⱼ) to this perturbation is determined as,

$${{R}_{ij} = \frac{d log {x}_{i}}{d log {p}_{j}} \vert}_{s t . st .} . \qquad ( 16 )$$

To infer the connection coefficients rᵢⱼ based on the experimentally measured, global responses Rᵢⱼ, the entire network is initially divided into n subnetworks, each containing only edges directed to a particular node (i). To determine the connection coefficients {rᵢₖ} for all xₖ (k≠i), n−1 independent parameters pⱼ (j=1, . . . , n−1) must be perturbed, neither of which can directly influence node i, whereas any other node k (k≠i) is affected by at least one of these parameters pⱼ. Formally, for each xᵢ (i=1, . . . , n), we choose a subset Pᵢof n−1 parameters pⱼ known to have the property that the function ƒᵢ for node i in Eq. 2 does not explicitly depend upon pⱼ, whereas each of the remaining nodes k (k≠i) is perturbed by at least one pⱼ∈Pᵢ. This condition is described as follows,

$$\frac{\partial {f}_{i} ( {x}_{1} , … , {x}_{n} , p )}{\partial {p}_{j}} = 0 , if k \neq i , then \frac{\partial {f}_{k} ( {x}_{1} , … , {x}_{n} , p )}{\partial {p}_{j}} \neq 0 at least for one {p}_{j} \qquad ( 17 )$$

Taken into consideration that rᵢᵢ=−1 (Eq. 15), all connections to the node i can be found by solving the following system of linear equations,

$${R}_{ij} = {\sum }_{k = 1 , k \neq i}^{n} {r}_{ik} {R}_{kj} , \frac{\partial {f}_{i} ( {x}_{1} , … , {x}_{n} , p )}{\partial {p}_{j}} = 0 , i = 1 , … , n ; j = 1 , … , n - 1 ; . \qquad ( 18 )$$

Repeating this procedure for all n subnetworks, the entire network is reconstructed.

This standard MRA procedure can fail when the data are too noisy or some module responses were not detected (Thomaseth et al., 2018). BMRA overcomes these limitations by explicitly incorporating noise in Eq. 18 (Halasz et al., 2016),

$${\sum }_{k = 1 , k \neq 1}^{n} {A}_{ik} {r}_{ik} {R}_{kj} + {ϵ}_{ij} = {R}_{ij} . \qquad ( 19 )$$

Here, Aᵢₖ are the elements of the adjacency matrix, which are equal to 1 if the connection coefficient rᵢₖ is non-zero, or equal to 0 otherwise; ϵᵢⱼ are the error variables assumed to be independently and identically distributed Gaussian random variables with the 0 mean and the variance σ², i.e. ϵᵢₖ˜ [symbol] (0, σ²). The error variance (σ²) is assumed to be a random variable with the inverse Gamma distribution, i.e. σ²˜IG(a, b), where a and b are the location and scale parameters. Following the common practice, we chose a=1, b=1. Further, for brevity we refer to this distribution P(σ²).

BMRA uses prior knowledge that is formulated in the form of the prior probability distributions. Based on the existing knowledge (Kanehisa et al., (2010). KEGG for representation and analysis of molecular networks involving diseases and drugs. Nucleic Acids Research 38, D355-D360; Szklarczyk D. et al., (2019). STRING v11: protein-protein association networks with increased coverage, supporting functional discovery in genome-wide experimental datasets. Nucleic Acids Research 47, D607-D613; Vaishnavi et al., 2015), we derived the reference network Aᵢ⁰={Aᵢₖ⁰}. The prior distribution P(Aᵢ), Aᵢ={Aᵢₖ}, has the maximum at the reference network Aᵢ⁰ and penalizes for the deviation from this network as follows, P(Aᵢ)∝exp(−ψ·d_{H}(Aᵢ, Aᵢ⁰)), where d_{H}(Aᵢ, Aᵢ⁰) is the Hamming distance between the network Aᵢ and the reference network Aᵢ⁰, ψ is a constant. The prior distribution of rᵢ is dependent on Aᵢ and σ² and is denoted by P(rᵢ|Aᵢ, σ²). If there is no direct connection from xⱼ to xᵢ, i.e. Aᵢⱼ=0, the corresponding connection strength (rᵢⱼ) is assumed to have 0 value with probability 1, whereas the connection strengths representing direct interactions (rᵢ={rᵢⱼ: Aᵢⱼ=1, j≠i}) were assumed to have a Gaussian prior P(rᵢ|Aᵢ)˜ [symbol] (0, Vᵢ) where Vᵢ=cσ²(RᵢR*ᵢ^{T}+λI). Here, R*ᵢ is the global response matrix of the nodes (nⱼ, j≠i) which directly regulate nᵢ (i.e. nⱼ, j≠i: Aᵢⱼ=1), c is the proportionality constant which is also known as the Zellner's constant. As previously, we chose c=Nₚⁱ, where Nₚⁱ is the number of perturbations other than those directly affecting node xᵢ, and λ=0.2 (Halasz et al., 2016; Santra et al., 2013).

Bayesian statistics is applied to update prior estimates of the binary vector Aᵢ={Aᵢₖ}, k=1, . . . , n and the vector of connection coefficients rᵢ={rᵢₖ}, k=1, . . . , n, to obtain posterior estimates of these variables using the experimental data, i.e., the global response matrix R=Rᵢₖ (Eq. 16),

$$P ( {r}_{i} , {A}_{i} , {σ}^{2} \vert R ) = \frac{P ( R \vert {r}_{i} , {A}_{i} , {σ}^{2} ) P ( {r}_{i} \vert {A}_{i} , {σ}^{2} ) P ( {A}_{i} ) P ( {σ}^{2} )}{P ( R )} . \qquad ( 20 )$$

Here, P(R|rᵢ, Aᵢ, σ²) is the likelihood function of the global response matrix R, given a connection coefficient vector rᵢ and a binary vector Aᵢ. P(rᵢ|Aᵢ, σ²) and P(Aᵢ) are the prior distributions of rᵢ and Aᵢ, respectively. The denominator P(R) is defined as follows,

$$P ( R ) = \int \int \int P ( R \vert {r}_{i} , {A}_{i} , {σ}^{2} ) P ( {r}_{i} \vert {A}_{i} , {σ}^{2} ) P ( {A}_{i} ) P ( {σ}^{2} ) {dr}_{i} {dA}_{i} d {σ}^{2} . \qquad ( 21 )$$

A key to BMRA is that the likelihood function for the observed global response matrix R is derived from the MRA equations (Eq. 18-20),

$$P ( R \vert {r}_{i} , {A}_{i} , {σ}^{2} ) = N ( {R}_{i} \vert {R}_{t}^{* T} r , {σ}^{2} I ) . \qquad ( 22 )$$

Here, Rᵢ={Rᵢₖ, k≠i} is the global response of node xᵢ to perturbations that do not directly affect xᵢ, R*ᵢ^{T} is the global response matrix of the nodes (xⱼ, j≠i) which directly regulate node xᵢ (i.e. xⱼ, j≠i: Aᵢⱼ=1, and r={rᵢⱼ: Aᵢⱼ=1, j≠i}. [symbol] N(Rᵢ|R*ᵢ^{T}r,σ²I) designates the normal distribution for Rᵢ where the mean equals R*ᵢ^{T}r and the variance σ²I.

The denominator in Eq. 20 that normalizes the probability P(rᵢ, Aᵢ, σ²|R) cannot be obtained analytically. The posterior distributions were estimated using Markov Chain Monte Carlo (MCMC) sampling algorithm. The posterior probability of Aᵢ provides a quantitative measure of how well a certain configuration of Aᵢ is supported by both prior knowledge and experimental data.

The values and confidence intervals for the corresponding connection coefficients are obtained from the posterior probability of rᵢ. To increase the accuracy of the method, we have modified the previously published algorithm (Halasz et al., 2016) and applied Occam's razor approach, by calculating mean and STD value of rᵢ using not the entire posterior distribution of rᵢ, but only the part that has the highest posterior likelihood.

### Preparation of the RPPA Perturbation Dataset and the BMRA Network Inference

Table S5C presents the list of analytes that are outputs of signaling modules (TRK, ERBB, ERK, AKT, JNK, S6K and RSK) of our core network. The output of the DPD module is determined using Eqs. 9-12 in the 70-dimensional molecular dataspace. To calculate the global response coefficients for the signaling modules, xᵢ, we used central fractional differences to approximate the logarithmic derivatives,

$${R}_{i j} = 2 \cdot \frac{{x}_{i 1} - {x}_{i 0}}{{x}_{i 1} + {x}_{i 0}} . \qquad ( 23 )$$

Here xᵢ₀ and xᵢ₁ are the i-th module outputs before and after a perturbation to the parameter pⱼ. Because the sign of the DPD value (S) could change for large perturbations, we used either left or right fractional differences,

$${R}_{s j} = \frac{{s}_{1} - {s}_{0}}{{s}_{0}} . \qquad ( 24 )$$

A feature of the BMRA formalism is that some modules might not be perturbed, but still the network topology will be inferred. In a core network we have not perturbed a module consisting of the ERBB family of RTKs, which can crosstalk with Trk receptors either directly or through downstream signaling pathways and feedback loops. The output of this additional RTK module (termed ERBB) is determined as the sum of EGFR and ERBB2 phosphorylation, measured with the corresponding antibody that does not distinguish between these two ERBB receptors. Having determined the global response coefficients of all other modules using Eqs. 23 and 24, BMRA inferred the connection strengths and confidence intervals that are given in Table S4.

### Using the Dynamic Phenotype Descriptor (DPD) to Model Global Network Control.

In order to understand how the core network exerts control over the global signaling network and specifies cell states and their transitions, we need to include it into the model. The STV/DPD formalism allows us to summarize all individual contributions to the global network responses in a dedicated network module termed the DPD.

This DPD module comprises all measured protein activities and abundances, except for the components of the core network. The absolute value of the DPD output (S) is the distance between the hyperplane that separates phenotypic states and the centroid of a point cloud of a given cell state (minus the core network components), but the sign of S can be negative or positive. This sign depends on whether the distance is determined in the parallel or antiparallel direction to the STV. For the selected STV direction, the sign of S is positive if a centroid of a point cloud is on the same side of the separation plane as a proliferation cloud and the sign of S is negative at the differentiation side. Therefore, any perturbation that drives the cellular response from differentiation to proliferation changes the S sign to positive, whereas moving from proliferation to differentiation makes S negative.

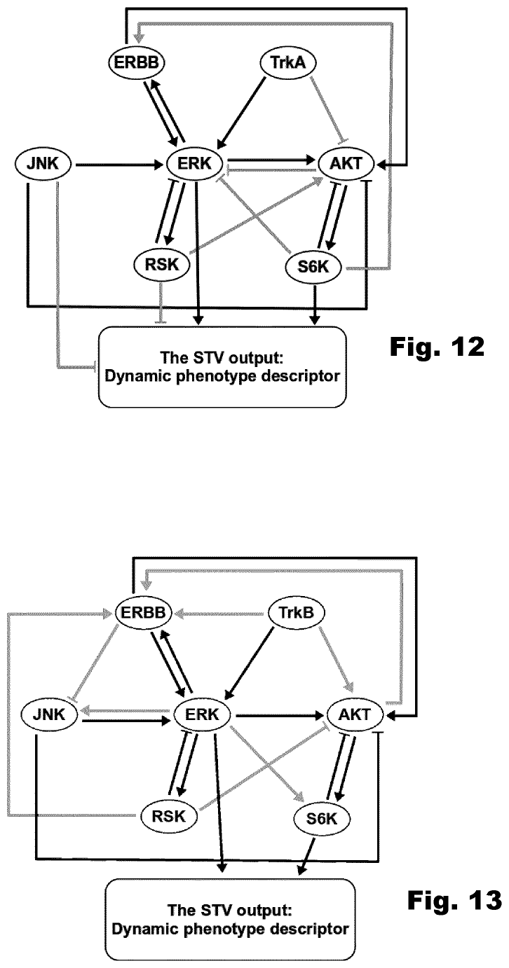

Introduction of the DPD allows us to systematically examine the influence of all core network pathways onto cell state transitions alone and in combination. We again used BMRA to determine connections to the DPD, as a network node, for each core pathway. The BMRA inferred influence of the core network on the DPD in TrkA and TrkB cells is shown respectively in FIGS. 12 and 13. Edges that are specific to TrkA and TrkB are shown in a lighter shade. The BMRA inferred influence of each signaling pathway on the DPD node is shown together with the reconstructed topologies of the core network.

As the DPD also links the network to cell fate decisions, the connection coefficient indicates not only a change in signaling but also a change in phenotype. A positive connection coefficient means that the cell is pushed towards proliferation, whereas a negative coefficient indicates a push to differentiation.

Because the changes in the DPD are downstream of the core network and therefore plausibly require more time, we assessed the DPD responses after 45 minutes of GF stimulation. By measuring the fold-changes in the outputs of signaling pathways and the STV module (ΔS/S), we obtained the global, systems-level responses to perturbations. These data enabled BMRA to infer the influence of each signaling pathway on the proximity of a point cloud to the separation hyperplane between proliferation and differentiation states, i.e., on the DPD and cell phenotype.

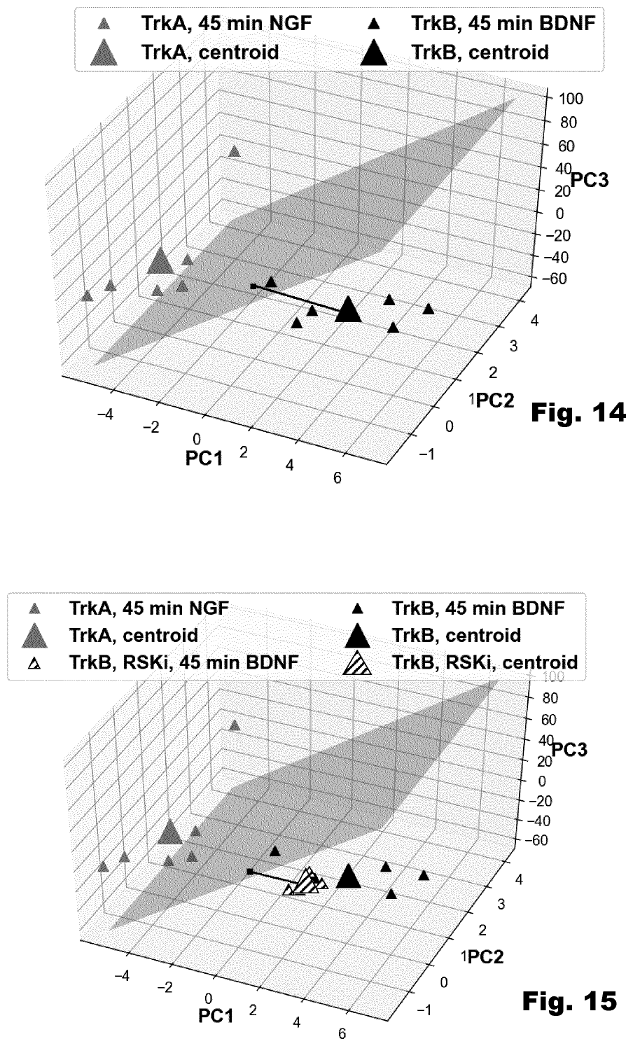

FIGS. 14 and 15 show PCA-compressed 45 min data points for TrkA cells (grey triangles), TrkB cells (black triangles), and, in FIG. 15, TrkB cells treated with p90RSK inhibitor, BI-D1870 (hatched triangles).

The distances of centroids of TrkB cells to the separation surface (light grey quadrilateral) before and after perturbation are shown by black lines. The DPD module output is the distance of a centroid from the separation hyperplane determined along or opposite the STV direction taken with the plus sign if the centroid is located at the right side from the separation hyperplane (proliferation), and with the minus sign if the centroid is at the left side (differentiation).

As expected, the ERK and the S6K modules strongly promote cell proliferation in both TrkA and TrkB networks. The facilitation of proliferation by RTKs, including Trk receptors, and AKT is mediated by their downstream effectors, i.e., by the ERK and S6K modules for RTKs and by the S6K module for AKT. At the same time, the influence of the RSK and JNK modules on cell phenotype is drastically different between these networks. In the TrkA network, RSK and JNK are two main signals that suppress proliferation and induce differentiation phenotype, whereas in the TrkB network these pathway modules do not influence the STV module and, therefore, the phenotype. Thus, ERK-induced activation of JNK and RSK modules in TrkB-expressing cells does not lead to the suppression of proliferation of these cells.

### Describing TrkA and TrkB Cell Signaling Dynamics by Mechanistic Modeling

Quantitative comprehension of signaling dynamics can only be achieved using a nonlinear mechanistic model. It is a necessary prerequisite to explain and predict how diverse experimental manipulations alter signaling patterns, resulting in changes in the DPD and, consequently, cell states. The BMRA-quantified core network topologies and the inferred influences of its signaling pathways on the DPD allow us to directly derive mechanistic dynamical models for TrkA and TrkB cells. These models predict both the dynamics of core pathway outputs and the changes in cellular phenotype.

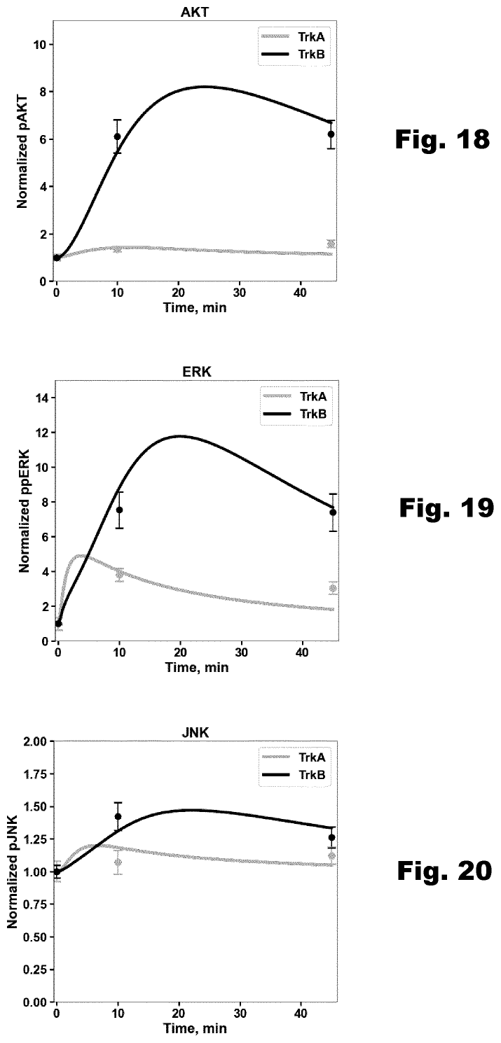

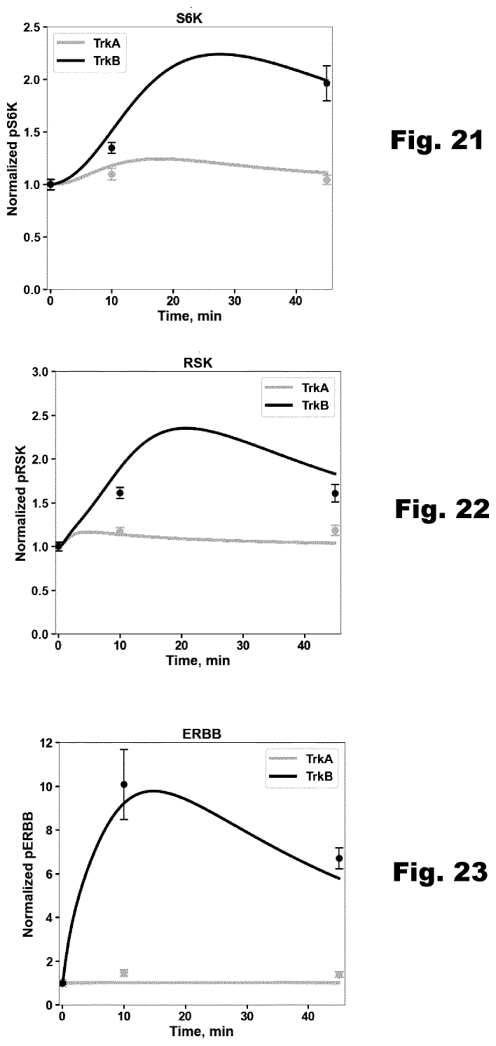

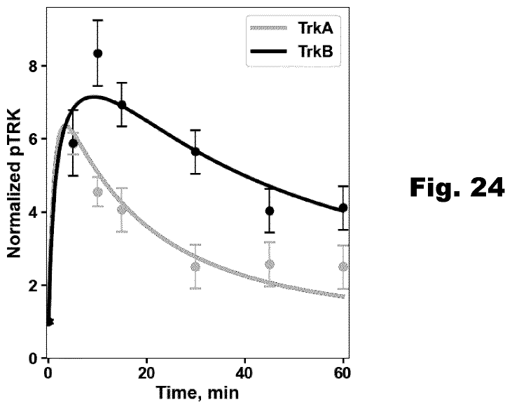

The TrkA and TrkB core networks showed several differences in connections and their strengths (FIGS. 10-13). The nonlinear kinetic models demonstrate that these alterations lead to distinct signaling patterns in these cells, which is supported by the data in FIGS. 18-24. In particular, based on the inferred activation of ERBB by TrkB and amplifying autocatalytic loops, ERBB->ERK->ERBB and ERBB->ERK->RSK->ERBB, the model predicts the higher and more sustained levels of active ERK in TrkB cells compared to TrkA cells, as seen in FIGS. 18-23.

FIGS. 18-24 show experimental data imposed on model predicted time-courses for TrkA (grey) and TrkB (black) cells stimulated with 100 ng/ml NGF and BDNF, respectively. Error bars represent SEM and are calculated using 3 biological replicates.

The model simulations, as seen in FIGS. 18-24, also show the higher and more sustained activation of RTKs, AKT, S6K and RSK activities in TrkB cells. The model correctly predicts not only responses of core network pathways of TrkA and TrkB cells to NGF and BDNF stimulation, but also their responses to different drug perturbations.

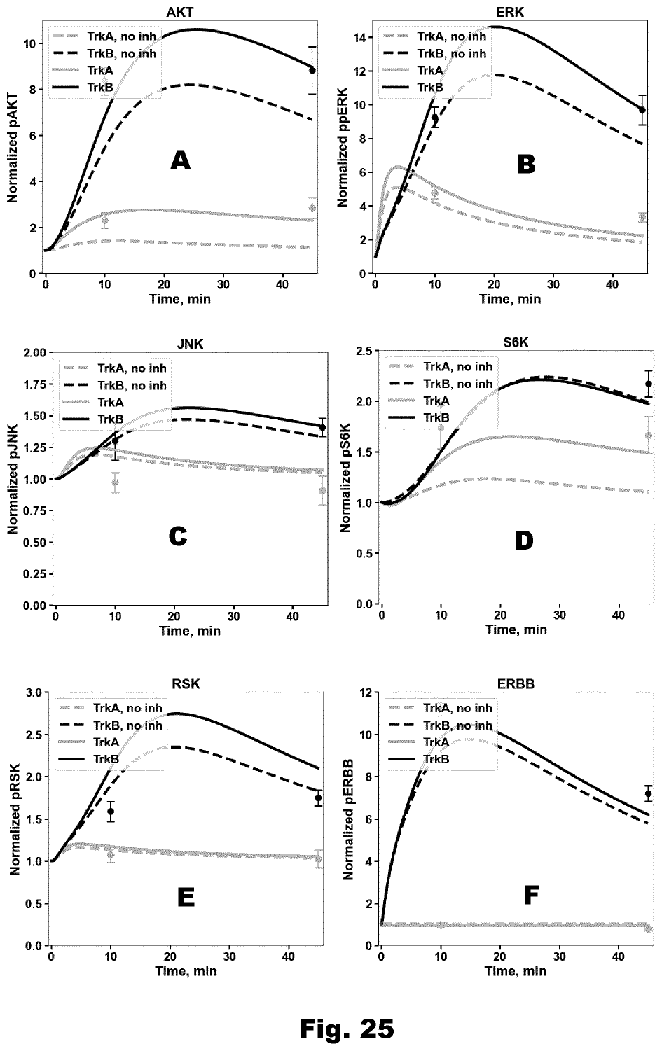

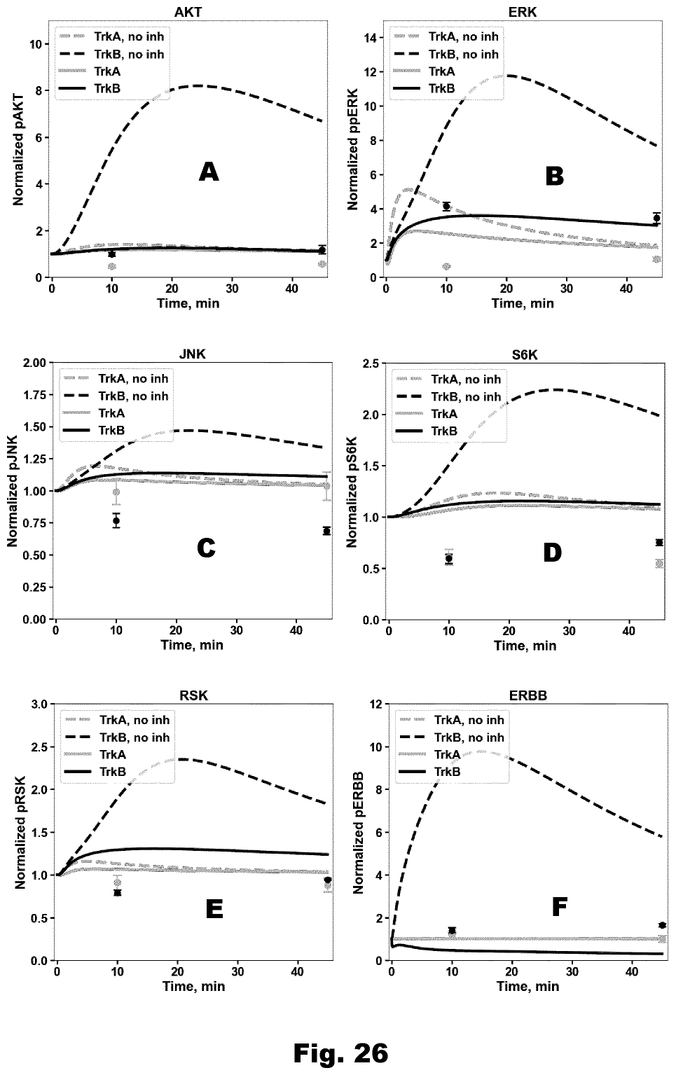

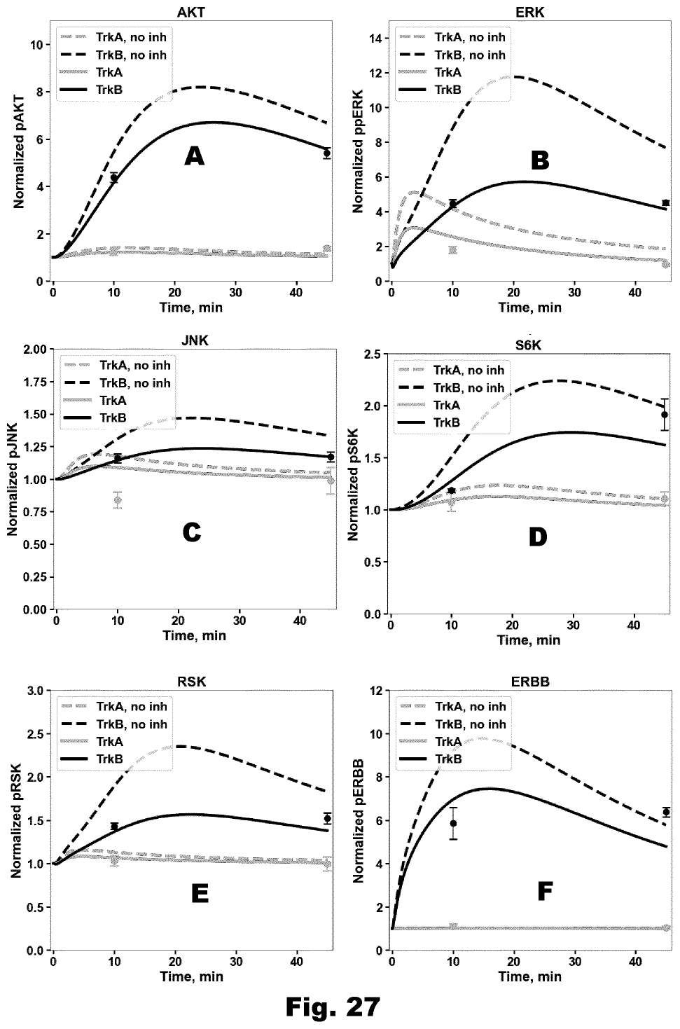

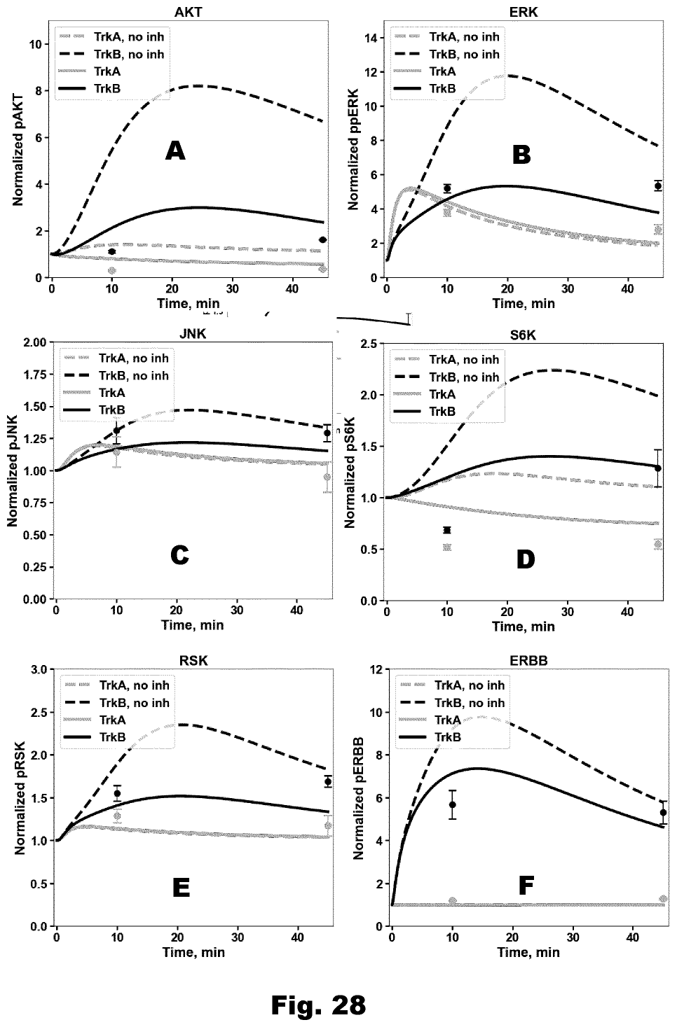

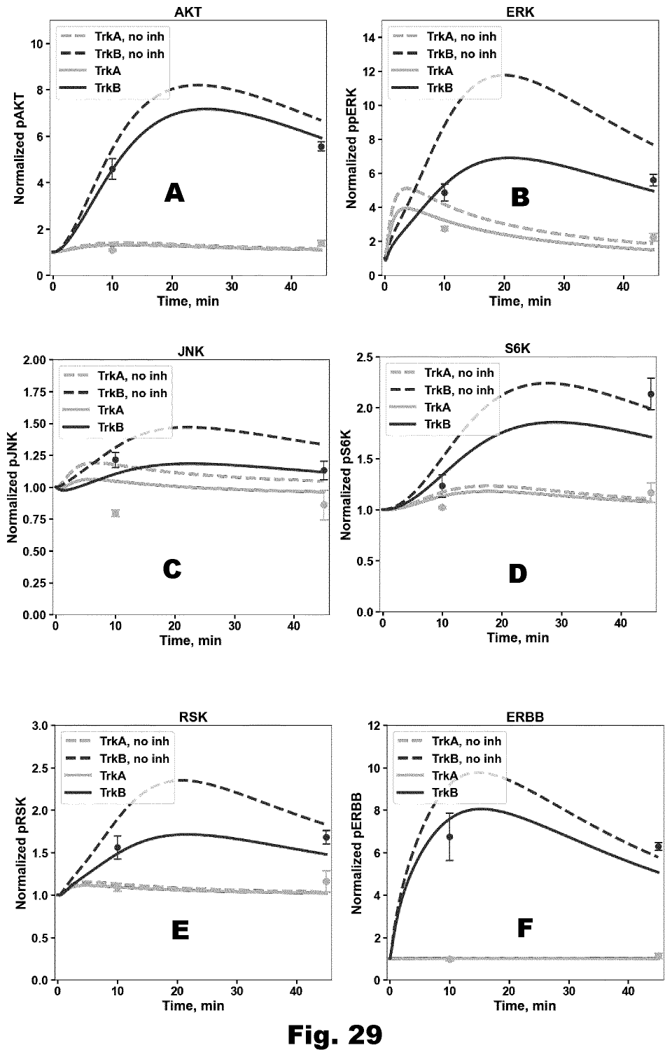

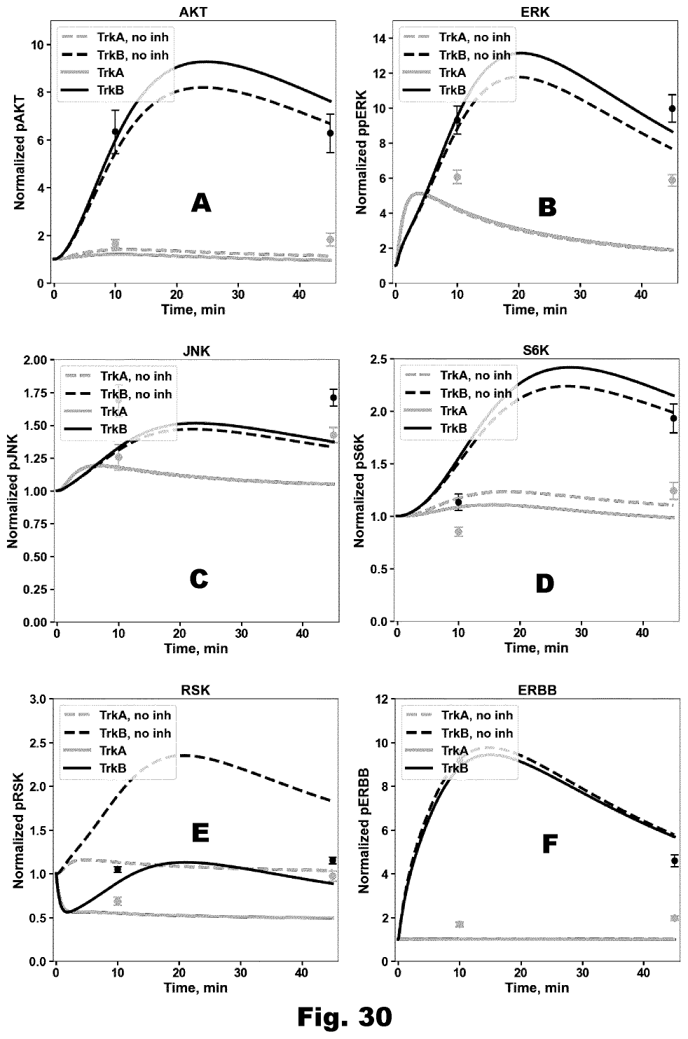

FIGS. 25-30 show the simulated time courses with the experimental data points (dots with error bars) imposed on the model predictions (curves) for a number of inhibitors: S6K inhibitor (1 μM) FIG. 25A-F; TRK inhibitor (5 μM) FIG. 26A-F; MEK inhibitor (0.5 μM) FIG. 27 A-F; AKT inhibitor (1 μM) FIG. 28A-F; JNK inhibitor (1 μM) FIG. 29A-F; and RSK inhibitor (1 μM) FIG. 30A-F. The TrkA and TrkB cells were treated with the respective inhibitor, and stimulated with 100 ng/ml NGF and BDNF, respectively. Dashed lines are the time courses in the absence of inhibitor. Error bars represent SEM and are calculated using 3 biological replicates.

For example, it can be seen that inhibition of S6K increases phosphorylation levels of ERK and AKT due to downregulation of S6K-induced negative feedback loops, which are stronger in TrkA cells than in TrkB cells (FIG. 25). Not surprisingly, both in TrkA and in TrkB cells inhibition of Trk receptors suppress signaling of every core pathway, confirming that their signaling are driven by Trk receptors (FIG. 26). Comparing responses to a MEK inhibitor in these cells demonstrates that even a moderate inhibition of ERK signaling substantially downregulates phosphorylation of ERBB, AKT, and their downstream effectors in TrkB cells, whereas in TrkA cells these effects are much less (FIG. 27). This is explained by the distinct wiring of core networks, emphasizing a key role of the positive feedback from the ERK module to ERBB in TrkB cells via RSK (FIGS. 12 & 13). Also, inhibition of AKT results in more pronounced suppression of core network signaling in TrkB than in TrkA cells due to positive feedback from AKT to ERBB specific to TrkB cells (FIG. 28). Thus, in TrkB cells both ERK and AKT pathways are involved in self-amplifying positive feedback loops to ERBB receptors, resulting in strong and prolonged stimulation of proliferation signaling. As we will see below, predictive simulations of these signaling patterns are extremely useful for reproducing and purposefully designing cell state changes following experimental manipulations.

### Methodology for Nonlinear Model of Core Signaling Network and Cell State Transitions

Using the quantified core network topologies and the inferred pathway influences on the DPD, a nonlinear ODE model is built by rule-based approach (Blinov, M. L. et al., (2004). BioNetGen: software for rule-based modeling of signal transduction based on the interactions of molecular domains. Bioinformatics; Borisov, N. M., et al. (2008). Domain-oriented reduction of rule-based network models. IET Syst Biol 2, 342-351; Chylek L. A. et al. (2014). Rule-based modeling: a computational approach for studying biomolecular site dynamics in cell signaling systems. Wiley Interdisciplinary Reviews: Systems Biology and Medicine 6, 13-36) for TrkA and TrkB cells. Here we describe the fundamentals of the model.

The activation of TRK and ERBB receptors by ligand binding and dimerization is modeled mechanistically. Briefly, NGF/BDNF binding to TrkA/TrkB is followed by receptor dimerization and phosphorylation, whereas the basal rate of ERBB dimerization is maintained by diverse GFs present in serum. The homo-dimerization of TrkA, TrkB and ERBB and hetero-dimerization of TrkB and ERBB is modeled using the thermodynamic approach developed previously (Kholodenko, B. N. (2015). Drug resistance resulting from kinase dimerization is rationalized by thermodynamic factors describing allosteric inhibitor effects. Cell Rep 12, 1939-1949). The binding of the first and second molecules of the ligand and the subsequent homo and hetero-dimerization of RTKs satisfy so-called “detailed balance” constraints (see, e.g., (Ederer and Gilles, (2007). Thermodynamically feasible kinetic models of reaction networks. Biophys J 92, 1846-1857; Hearon, J. Z. (1953). The kinetics of linear systems with special reference to periodic reactions. Bull Math Biophys 15, 121-141; Kholodenko et al., (1999). Quantification of short term signaling by the epidermal growth factor receptor. J Biol Chem 274, 30169-30181).

These thermodynamic restrictions require the product of the equilibrium dissociation constants (K_{d}'s) along a cycle to be equal to 1, as at equilibrium the net flux through any cycle vanishes, since the overall free energy change is zero. Because ligand binding facilitates the RTK dimerization, following the thermodynamic approach (Kholodenko, 2015), we introduce three thermodynamic factors, describing how the K_{d}'s of homo- and hetero-dimerization of RTKs change upon ligand binding. When Trk receptor inhibitor is added, an inhibitor-free protomer can still cross-phosphorylate the other protomer in a dimer.

The core network dynamics is modeled up to 45 minutes, and therefore the total moieties of ERK, AKT, JNK, S6K and RSK are assumed to be conserved. However, internalization of RTKs that is occurring on this timescale is included in the model (Cosker, K. E., and Segal, R. A. (2014). Neuronal signaling through endocytosis. Cold Spring Harb Perspect Biol 6). Following, internalization RTKs are subsequently degraded, whereas there is also an influx of receptors from the cell interior to the membrane. The disappearance of RTKs from the plasma membrane depends on the dimer composition. In the model the rate of internalization of TrkB-ERBB heterodimers is assumed to be slower than the internalization rate of TrkA and TrkB homodimers, based on the literature. The BMRA-inferred connections show that there are multiple feedback loops to the ERBB module from downstream kinase modules (Table S4). The influence of these feedbacks on the ERBB module activity is modeled as hyperbolic multipliers that modify the rate of activating ERBB phosphorylation (Tsyganov, M. A. et al. (2012). The topology design principles that determine the spatiotemporal dynamics of G-protein cascades. Mol Biosyst 8, 730-743). The RTK dephosphorylation is catalyzed by phosphatases. The activation and deactivation dynamics of the downstream signaling modules is modeled using the Michaelis-Menten kinetics and hyperbolic multipliers that account for signaling crosstalk between the pathways. The developed model of the core signaling network consisted of 81 species and 404 reactions.

The BMRA network reconstruction constrains parameters of the dynamical model by maximum likelihood values of the inferred connection strengths (Table S4). In particular, only interactions between modules where the connection coefficients have statistically significant non-zero values are included in the model. Additional constraints on the parameter values occur because the inferred connection coefficients are normalized Jacobian elements (Kholodenko et al., 2002), which are functions of the model parameters (Eq. 15 and as described further below).

The model includes the DPD module whose output summarizes the contributions of all individual proteins (minus core network constituents) to the global network responses. This module describes cell-wide signaling and the DPD output (S) is defined by Eq. 9. The DPD maps the network-wide changes, which occur in the multidimensional molecular dataspace upon perturbations, into a 1D (S) space. If the data point clouds before and after a particular perturbation are measured in the experiments, ΔS can be calculated using Eq. 12. Our model allows to determine the dynamics of S following any drug perturbation to core network pathways. The calculated DPD trajectory is a 1D projection of cell maneuvering in Waddington's landscape, determined as follows,

$$\frac{dS}{dt} = f ( S ) + {\sum }_{j} {r}_{sj} ( \frac{{S}_{st . st .}}{{x}_{j}^{st . st}} ) {x}_{j} ( t ) \qquad ( 25 )$$

Here ƒ(S) is the restoring driving force guided by Waddington's landscape. The sum in Eq. 25 is the signaling driving force, xⱼ(t) are the outputs of signaling modules, r_{Sj} are the corresponding, BMRA-inferred connection coefficients to the STV (see Table S4), and S_{st.st.} and xⱼ^{st.st} are the initial steady-state values of S and x; before perturbations.

The restoring driving force ƒ(S) is given by the derivative of the potential (U), as follows

$$f ( S ) = - \frac{d U}{d S} \qquad ( 26 )$$

The potential (U) that models Waddington's landscape has 3 minimums. These minimums correspond to three stable steady states of neuroblastoma cells: the ground state (Sg), differentiation (S_{d}) and proliferation (Sₚ). There are two unstable steady states at the borders between the basins of attraction of two neighboring steady states.

Assuming the quadratic potential U in the vicinity of each stable state, (which is widely used in physics (Landau and Lifshitz, 1980)), the restoring driving force ƒ(S) is modeled using a piece-wise linear approximation. This force ƒ(S) is set to zero at the borders between the basins of attraction, and ƒ(S) reaches its maximum at the half distance to the border from the stable steady state (Eq. 27).

$$f ( S ) = \{ \begin{matrix}- α ( S - {S}_{0} ) , & S < \frac{3 {S}_{0} + {S}_{D}}{4} \\ α ( S - \frac{{S}_{0} + {S}_{D}}{2} ) , & \frac{3 {S}_{0} + {S}_{D}}{4} < S < \frac{3 {S}_{D} + {S}_{0}}{4} \\ - α ( S - {S}_{D} ) , & \frac{3 {S}_{D} + {S}_{0}}{4} < S < \frac{3 {S}_{D} + {S}_{P}}{4} \\ α ( S - \frac{{S}_{P} + {S}_{D}}{2} ) , & \frac{3 {S}_{D} + {S}_{P}}{4} < S < \frac{3 {S}_{P} + {S}_{D}}{4} \\ - α ( S - {S}_{P} ) , & S > \frac{3 {S}_{P} + {S}_{D}}{4}\end{matrix} \qquad ( 27 )$$

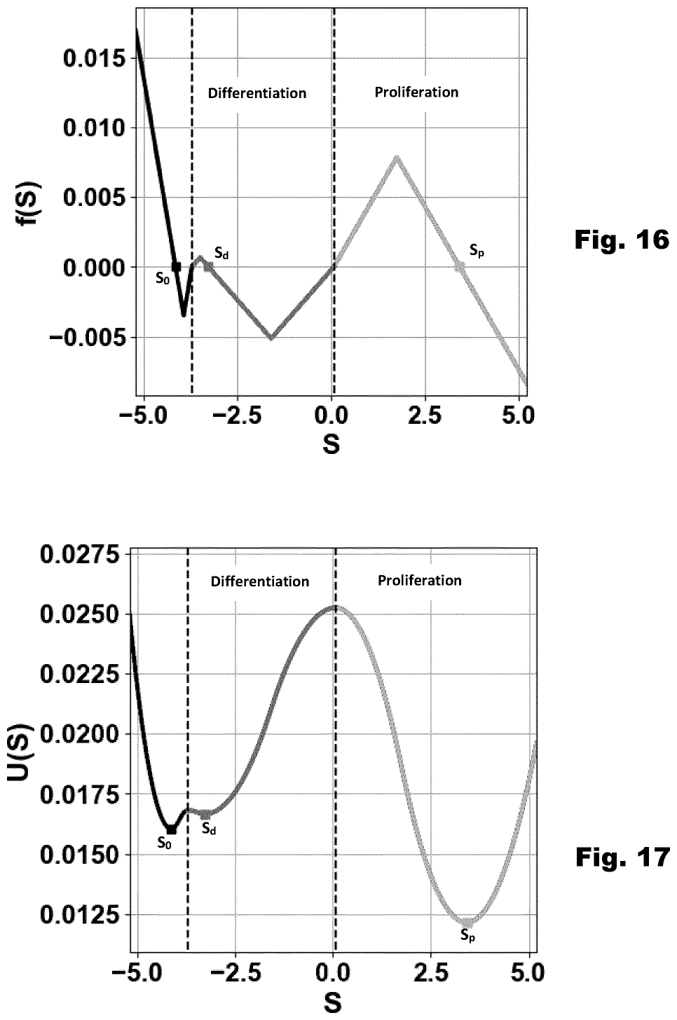

FIG. 16 shows the restoring driving force, while FIG. 17 shows the corresponding Waddington's landscape potential. Three local minima of Waddington's landscape correspond to centroids of the three cell states: ground (S₀), differentiation (S_{d}) and proliferation (Sₚ). A cell's movement in the landscape is guided by the signaling driving force and the restoring driving force.

Eq. 25 allows for an interpretation of a cell progressing through the molecular dataspace as a particle that moves in the potential force field (Waddington's landscape) and the field of external forces exerted by responses of core signaling pathways,

$$\frac{d S}{d t} = - \frac{d U}{d S} + {\sum }_{j} {r}_{sj} ( \frac{{S}_{st . st .}}{{x}_{j}^{st . st}} ) {x}_{j} ( t ) \qquad ( 28 )$$

In the vicinity of the steady state Sᵢ∈{S₀, S_{D}, S_{P}}, the solution of Eqs. 26 and 27 is expressed analytically as follows,

$$S ( t ) = {S}_{i} + {e}^{- α t} {\int }_{0}^{t} ( {\sum }_{j} {r}_{sj} ( \frac{{S}_{st . st .}}{{x}_{j}^{st . st}} ) {x}_{j} ( τ ) ) {e}^{α τ} d τ \qquad ( 29 )$$

Eq. 29 illustrates the system has the characteristic memory time, tₘ˜1/α. On the times much smaller than the memory time, t<<tₘ, the entire change in S is determined by the time integral over signaling driving force.

### Refining Parameters of the Dynamic Model

To decrease the number of parameters to fit, the concentrations of different protein forms and the parameters with the concentration dimensionality, such as, the Michaelis' constants, were normalized on the conserved total protein concentrations. Only the time was left as the dimensional variable (measured in seconds) to readily interpret model simulations.

To refine the parameters of pathway interactions of our core network inferred by BMRA, the data were split into a training set and a validation set. The training set included the time course of TrkA and TrkB phosphorylation measured by Western Blot and 10 min RPPA data for the remaining signaling modules. The model-generate time courses were fitted to these training set data with the objective function defined as the sum of squares of deviations. A feature of our parameter refinement is that in addition to the training dataset, we constrained the parameters using the BMRA inferred connection coefficients within their confidence intervals. Implicit constraints on the parameter values occur because the connection coefficients defined in Eq. 15 have to be within the confidence intervals of the BMRA inferred connections. Then, we used a unique feature of the pyBioNetFit software, which allows adding parameter constraints in the forms of inequalities to the parameter fitting process (Mitra et al., 2019). A combination of scatter search and simplex methods and pyBioNetFit software were used to fit the model simulations to the training dataset, as shown in FIGS. 18-30. Scatter search with population size 20 was used to obtain the initial parameter set, and simplex algorithm was used for the local refinement of the initial set. The validation set consisted of 45 min RPPA data for signaling modules of the core network.

In total, the rule-based nonlinear model of the core signaling network and cell state transitions consists of 82 species and 405 reactions. The simulations of the models were run using BioNetGen software (Blinov et al., 2004), which used CVODE routine from the SUNDIALS software package (Hindmarsh, A. C. et al., (2005). SUNDIALS: Suite of nonlinear and differential/algebraic equation solvers. ACM Trans Math Softw 31, 363-396) for solving ordinary differential equations (ODE). Matplotlib Python package was used for plotting experimental and modeling results.

Eq. 25 determines the DPD dynamics when the cell progression through the molecular dataspace is directed by the signaling driving force and the restoring force. For the signaling driving force, we fit the coefficients,

$${β}_{j} = {r}_{s j} ( \frac{{S}_{st . st .}}{{x}_{j}^{st . st}} ) ,$$

in the ranges constrained by the confidence intervals of the BMRA-inferred connection coefficients (r_{Sj}), whereas the signaling module outputs (xⱼ) are calculated by the model. For the restoring driving force, Eq. 27, only slopes parameters (α) are fitted.

### A Mathematical Description of the Forces Guiding Cell Motion in Waddington's Landscape.

A key feature of the cSTAR approach is that we integrate cell state transitions into a mechanistic kinetic model. Thus, we follow both the kinetics of pathway outputs of the core network (FIGS. 18-30) and the changes in cellular phenotype. This combination allows us to map how a cell maneuvers in a Waddington landscape and how external perturbations can manipulate this journey.

In the molecular dataspace, we present cell states as centroids of the data point clouds that describe a particular phenotype. Before ligand stimulation or drug perturbations, cells reside in (meta)stable states. Following a perturbation, the movement of a centroid is governed by two driving forces. One is a signaling driving force that emerges from the changes in core network activities, and the other is a restoring force that pushes the centroid back to its original (meta)stable state, provided the deviation from this original state has not been too large (FIGS. 16 and 17). Only the pathways with non-zero connections to the STV module generate signaling driving force that affects the DPD. We have determined this force using the BMRA inferred pathway influence on the DPD (Table S4 and FIGS. 12 and 13). The restoring driving force initially increases in the vicinity of the original cell state but then decreases to zero at the cell state separation surface (FIG. 16). In biology, the restoring driving force is determined by Waddington's landscape (Brackston et al., 2018; Lu et al., 2014; Wang et al., 2011) and in physics by the free energy landscape (Haken, 2004; Landau and Lifshitz, 1980). Yet, in both disciplines, the restoring driving force specifies how a system evolves in the absence of external perturbations. Consequently, a system state will change either due to external perturbations pushing the system over the activation barrier or due to internal or external noise that might result in a spontaneous overpassing of the barrier. Summarizing, a key distinction of the cSTAR approach is that we introduce a signaling driving force coming from signaling responses to drug perturbations and imposed on the initial Waddington's landscape (FIG. 17).

### Maneuvering in Waddington's Landscape.

The mechanistic nature of the BMRA based network reconstruction combined with the inclusion of the DPD as a quantitative indicator of cell states, allows us to calculate the DPD values following GF stimulation.

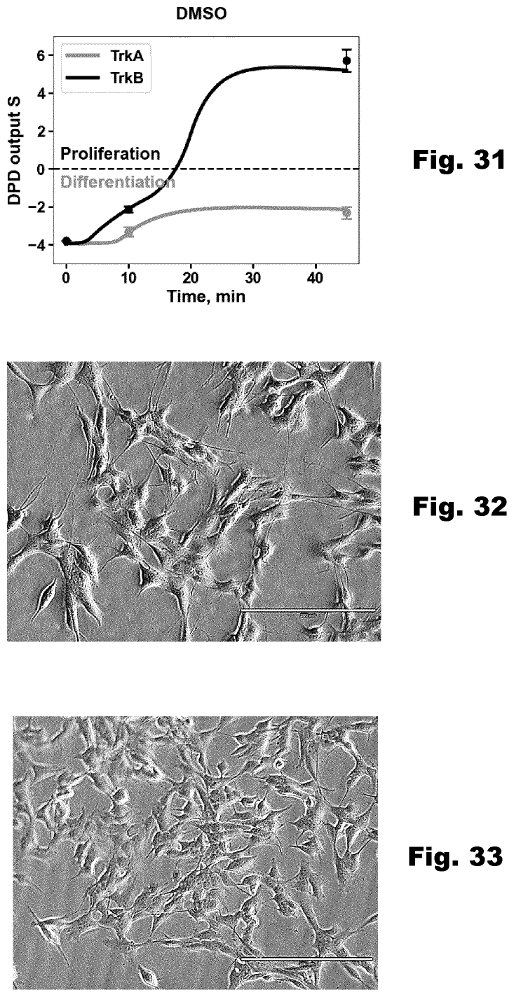

Our simulations show that starting from the ground state TrkA and TrkB cells differentially progress through Waddington's landscape and assume two different states, differentiation and proliferation, as shown in FIG. 31, which shows experimentally measured (dots) and model-predicted (solid lines) responses of DPD output S to ligand stimulation in TrkA and TrkB cells. Error bars are calculated using 3 biological replicates.

These predictions are further supported by the experimental data illustrated in FIGS. 32 and 33. FIGS. 32 and 33 are live cell images of TrkA and TrkB cells stimulated with GFs for 72 hours (TrkA+NGF; TrkB+BDNF), and showing differentiation of TrkA cells in FIG. 32 and proliferation of TrkB cells in FIG. 33.

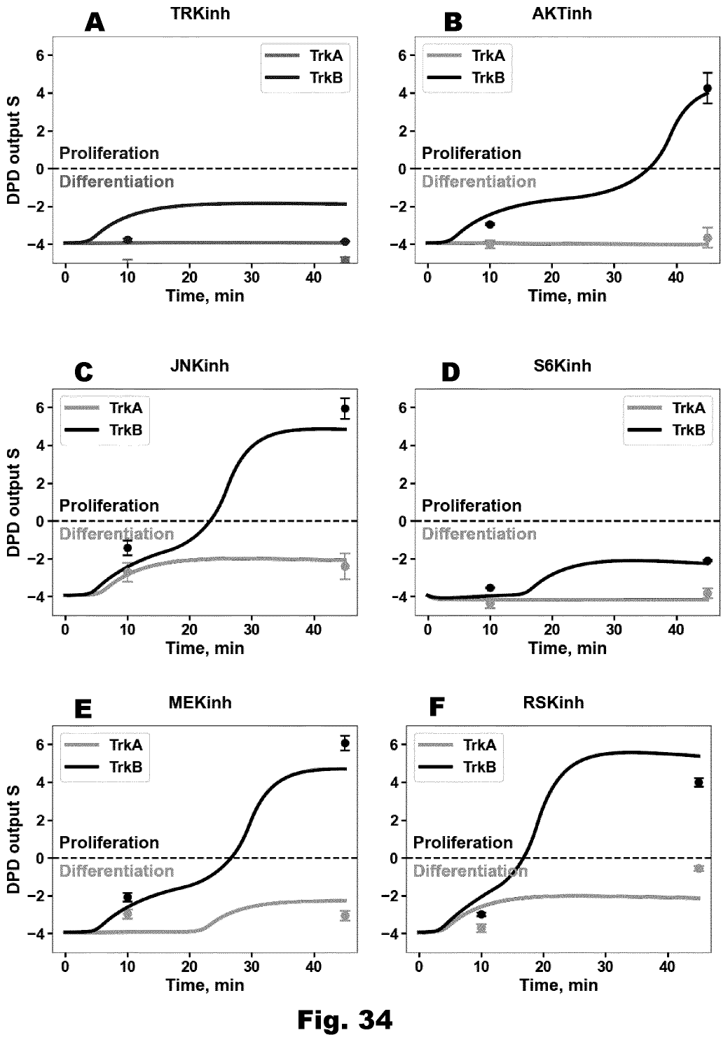

We also calculated the DPD trajectories after inhibitor perturbations. FIG. 34A-F shows model-predicted time courses (solid lines) and experimentally measured (dots) DPD responses of TrkA (grey) and TrkB (black) cells to diverse inhibitor perturbations. These calculations readily reiterate first approximation predictions made by using experimental data for dot products of the STV and perturbations vectors (Tables 1 and S5).

The model shows that inhibition of TrkB and S6K are pro-differentiation interventions (FIGS. 34A and 34D), whereas inhibition of RSK in TrkA cells interferes with differentiation (FIG. 34F).

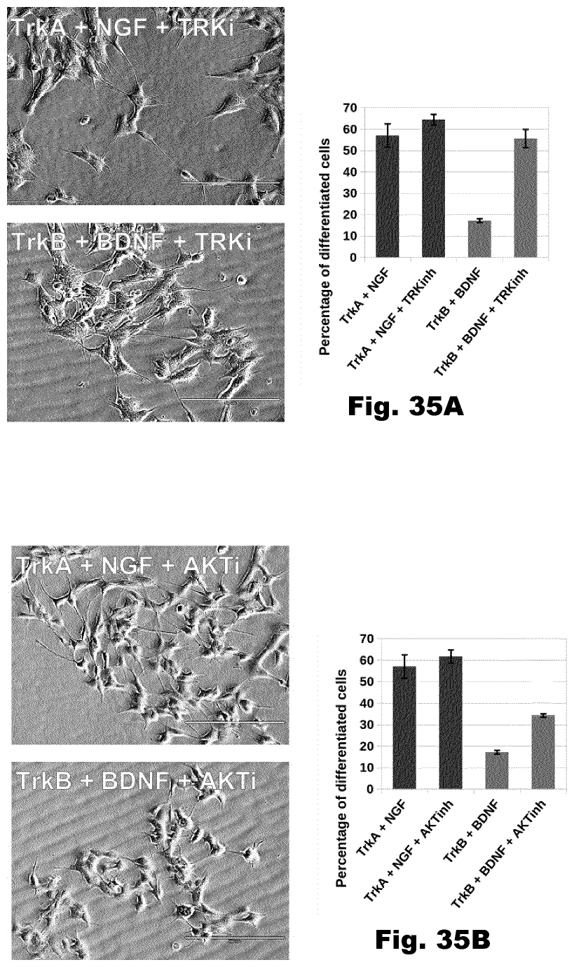

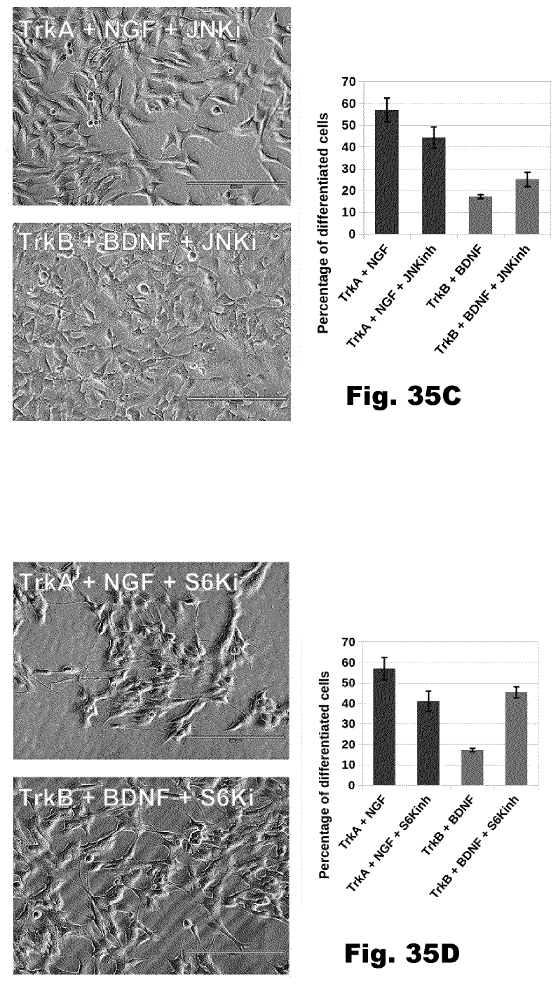

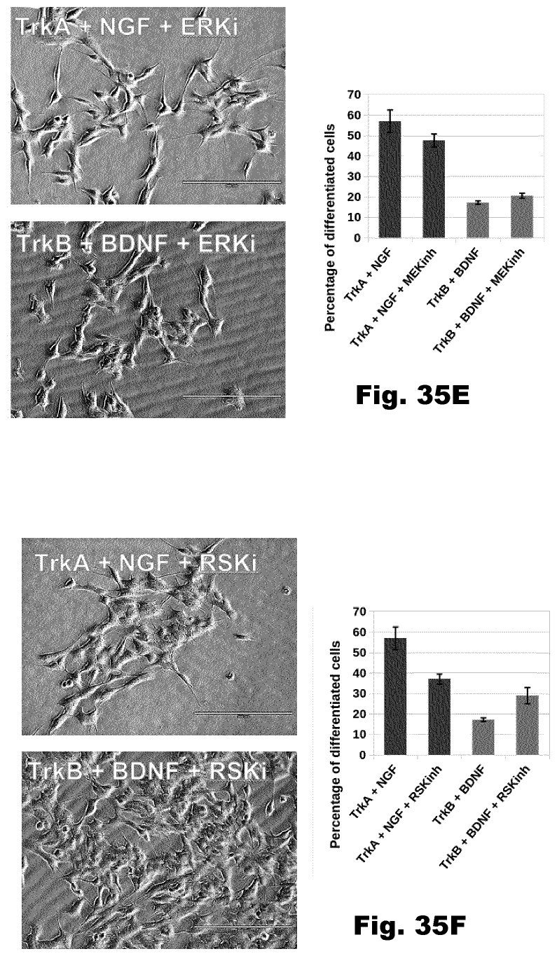

These simulations are corroborated by live cell images taken at 72 hours in these cells (FIGS. 35 A-F). Inhibitor concentrations are the same as for FIGS. 25-30. In each of these images, the scale bar shown in the lower right denotes a distance of 200 micron, as it is for all other images in the drawings where a scale bar is visible.

In FIGS. 35A-F, the live cell images are accompanied by bar plots showing the percentage of differentiated cells. The percentages were obtained by examining three images for each set of conditions, and counting the total number of cells and the number of differentiated cells.

Interestingly, the model shows that whereas RSK does not directly affect the STV module output in TrkB cells, RSK inhibition decreases the ERBB module activity, resulting in the decrease of proliferation stimulation by the ERK and S6K modules (Table S5 and FIG. 34F).

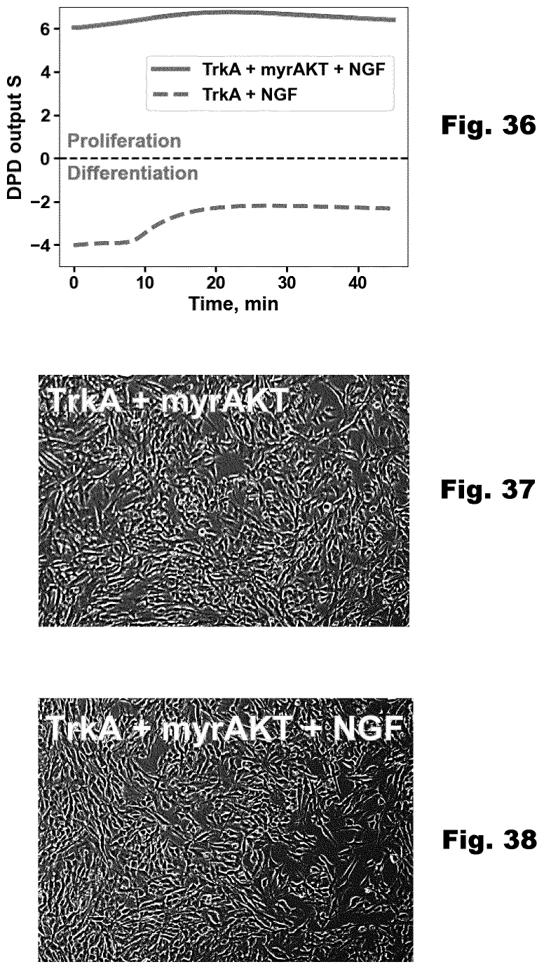

Thus, the developed model allows capturing both direct and network-mediated effects of drugs on cell phenotype. The model predicts that a marked increase in the AKT activity will result in abolishing differentiation and increased proliferation of TrkA cells. This is illustrated in FIG. 36.

In FIG. 36, the simulated DPD time course of NGF-stimulated TrkA cell response to the 10-fold increase in the Vmax of AKT activation predicts persistent proliferation (solid line), whereas the simulated DPD time course of NGF-stimulated control cells shows a switch to differentiation (dashed line).

Indeed, transfection of TrkA cells with myristolated AKT, which is constantly active, stops differentiation and leads to proliferation of TrkA cells. FIGS. 37 and 38 are live cell images of TrkA cells transfected with myristoylated AKT 72 before and after stimulation with NGF for 72 hours.

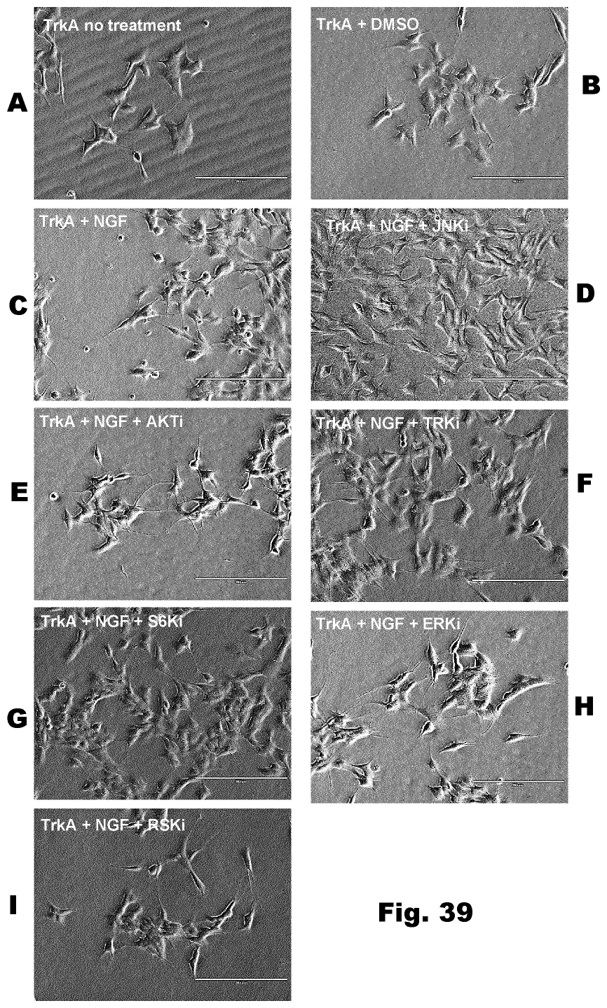

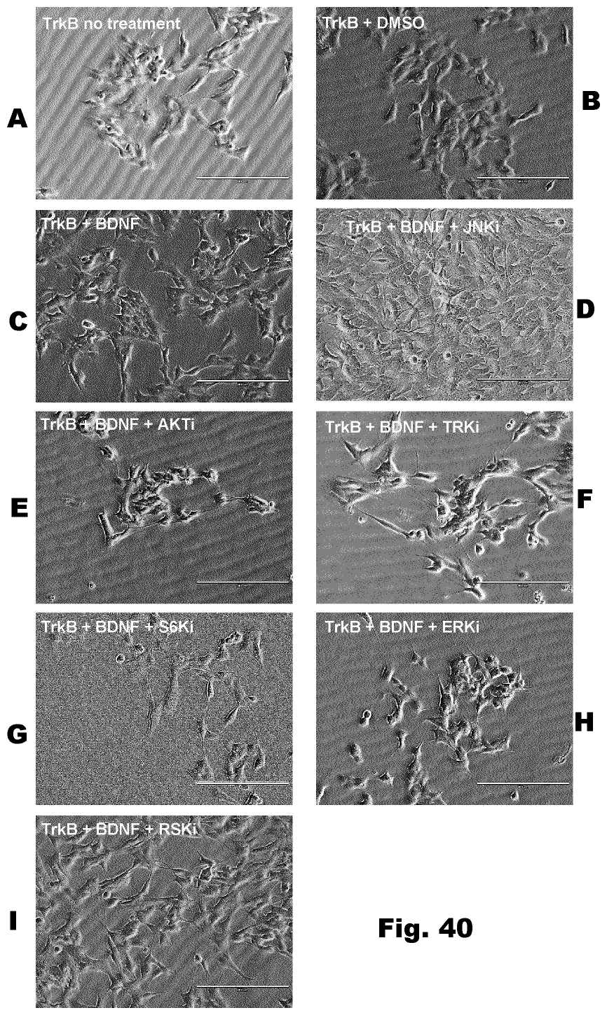

Although the sensitivity of the STV module output to diverse signaling inhibitors (Trk, S6K, ERK, AKT, RSK and ERBB) is different, simulations show that sufficiently high doses of these inhibitors facilitate, at least partially, TrkB cell differentiation (FIG. 34A, B, D-F), which is supported by the experimental observations in FIG. 35. Additional replicates of TrkA and TrkB cell images for all inhibitor perturbations are given in FIGS. 39A-I (which shows live cell images of TrkA cells stimulated with NGF and treated with inhibitors at the same concentrations as previously detailed) and 40A-I (which shows live cell images of TrkB cells stimulated with BDNF and treated with inhibitors at the same concentrations as previously detailed).

Using the model, we can calculate not only time dependent DPD responses to a certain drug but also DPD dose responses. Importantly, the model predicts signaling patterns and cell state responses for different doses of drugs applied not only separately but also in combinations.

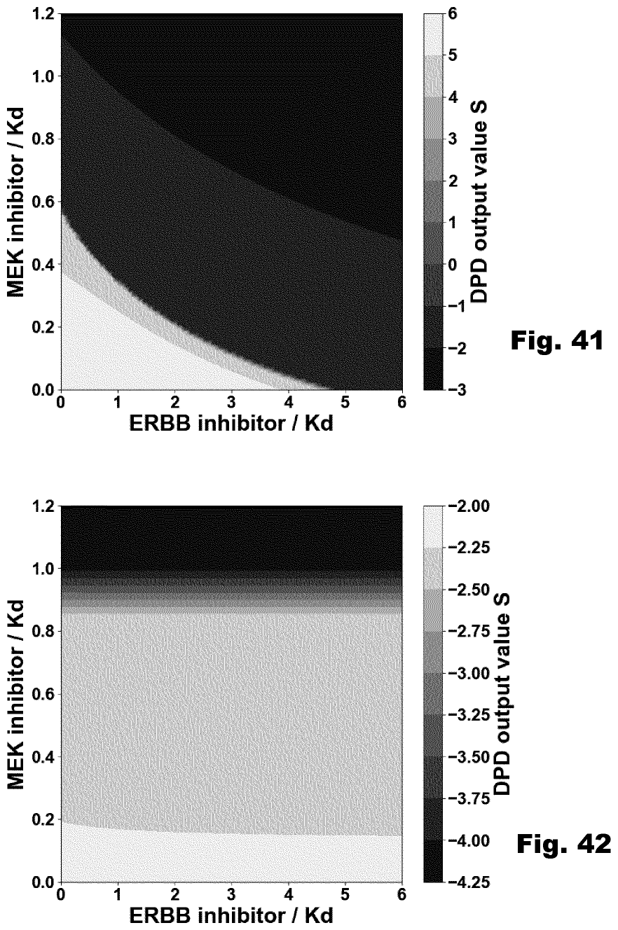

FIGS. 41 and 42 show predictive simulations of how a combination of ERBB and ERK inhibitors will change the DPD in TrkB and TrkA cells.

In FIG. 41, the model-predicted DPD responses of TrkB cells to ERBB and ERK inhibitors applied separately and in combinations are shown at 45 min 100 ng/ml BDNF stimulation using Loewe isoboles. Concave isoboles demonstrate synergy.

In FIG. 42, the model-predicted DPD responses of TrkA cells to ERBB and ERK inhibitors applied separately and in combinations are shown at 45 min 100 ng/ml NGF stimulation using Loewe isoboles. The ERBB inhibitor applied alone has negligible effect.

The model suggests that this combination synergistically induces differentiation of TrkB cells (FIG. 41), whereas it does not change the state of TrkA cells (FIG. 42).

Experiments corroborate model predictions, showing that a combination of the ERBB inhibitor Gefitinib and the MEK inhibitor Trametinib synergistically inhibits ERK signaling.

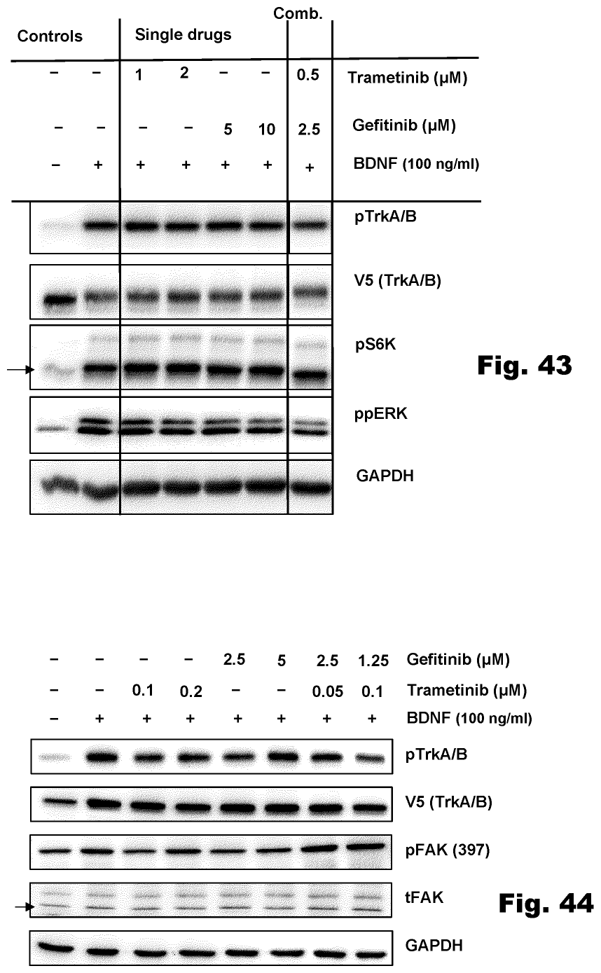

FIG. 43 shows responses of ERK (ppERK) and p70S6K (pS6K) phosphorylation to Geftitinib (ERBB family inhibitor, applied at 5 and 10 μM), Trametinib (MEK inhibitor, 1 and 2 μM) and their combination (2.5 μM and 0.5 μM) at 45 min in TrkB cells.

FIG. 44 shows responses of FAK phosphorylation (a marker of cell differentiation) to Geftitinib (2.5 and 5 μM), Trametinib (0.1 and 0.2 μM) and their combination (2.5 and 0.05 μM, and 1.25 and 0.1 μM) at 72 hours. This inhibitor combination has synergistically induced the FAK phosphorylation, which is a well-established differentiation marker (Dwane, S. et al. (2013). Optimising parameters for the differentiation of SH-SY5Y cells to study cell adhesion and cell migration. BMC Research Notes 6, 366).

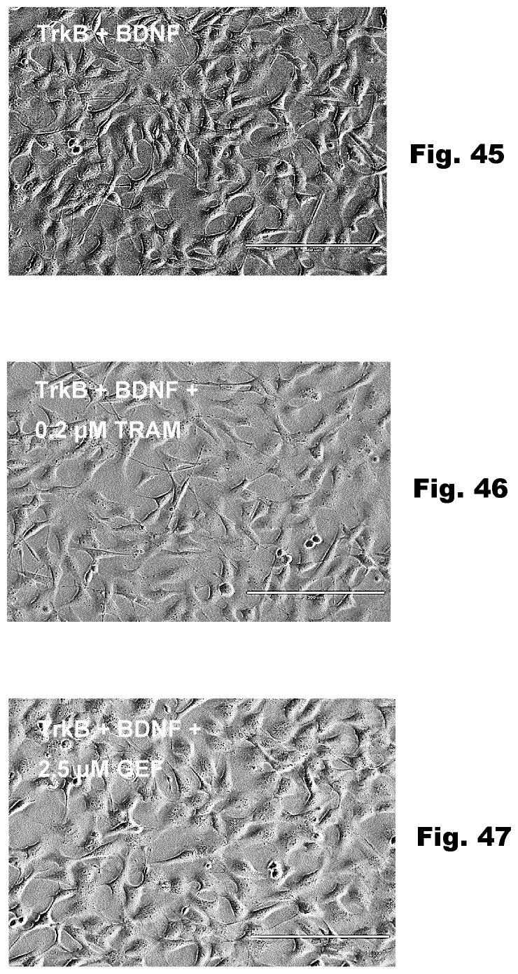

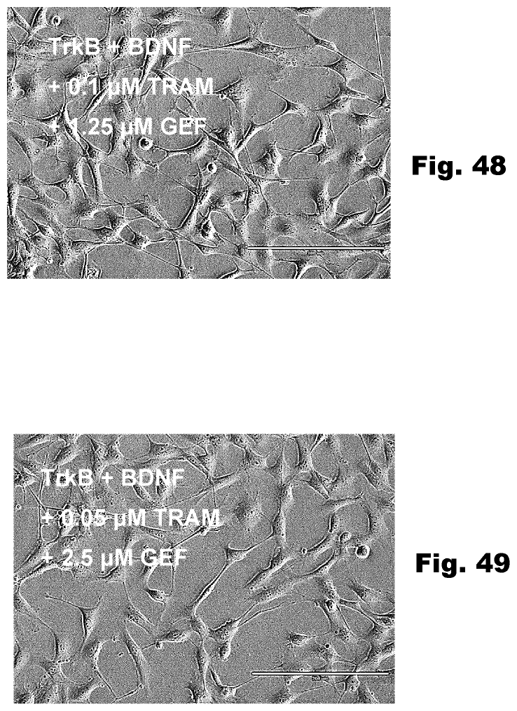

FIG. 45 is a live cell image of TrkB cells stimulated with BDNF taken at 72 hours. FIG. 46 is a live cell image of BDNF-stimulated TrkB cells treated with 0.2 μM Trametinib taken at 72 hours. FIG. 47 is a live cell image of BDNF-stimulated TrkB cells treated with 2.5 μM Gefitinib taken at 72 hours. FIG. 48 is a live cell image of BDNF-stimulated TrkB cells treated with a combination of 1.25 μM Gefitinib and 0.1 μM Trametinib taken at 72 hours. FIG. 49 is an additional experiment, being a live cell image of BDNF-stimulated TrkB cells treated with a combination of 2.5 μM Gefitinib and 0.05 μM Trametinib taken at 72 hours.

FIGS. 45-49 show that a combination treatment with Gefitinib and Trametinib, but not with either inhibitor applied separately in a 2-fold higher dose than in combination, produced marked differentiation of TrkB cells.

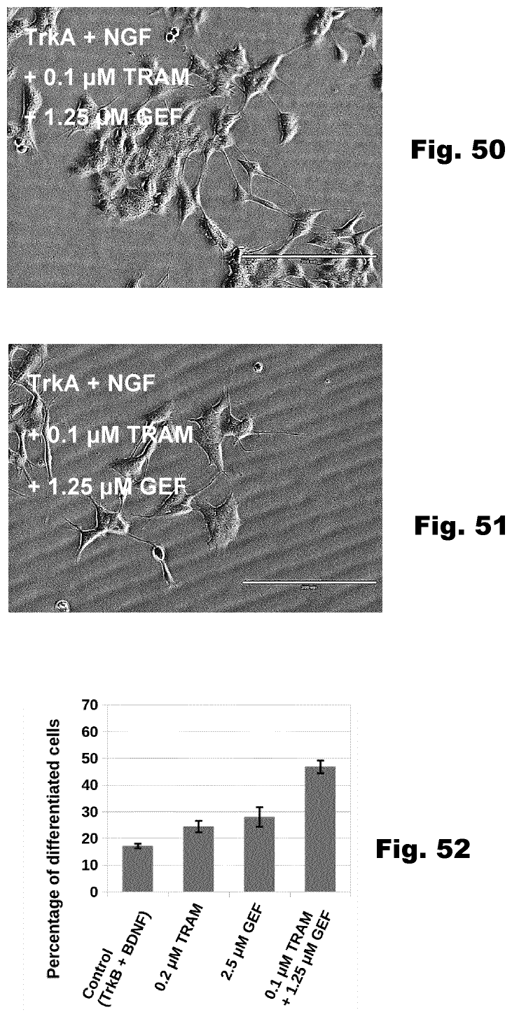

FIGS. 50 and 51 are live cell images of NGF-stimulated TrkA treated with a combination of 1.52 μM Geftitinib and 0.1 μM Trametinib taken at 72 hours. FIGS. 50 and 51 demonstrate that this same combination did not change TrkA cell states.

FIG. 52 is a bar plot showing the percentage of differentiated cells for different treatments, measured by counting cells in images and calculating the ratio of differentiated cells to total cells.

cSTAR Flexibility and Scalability

Next, we tested cSTAR's performance with data of different type and scale. Using the same conditions as in the RPPA dataset, we acquired quantitative phosphoproteomics MS datasets for TrkA and TrkB cells. Calculating the STV and DPD changes for cell-wide signaling pattern of ca. 5000 phosphosites resulted in similar core network components and a key prediction of synergy between ERBB and MEK inhibitors in inducing TrkB cell differentiation without affecting the TrkA cell phenotype (FIG. 6D, Supplementary Information), which was experimentally validated.

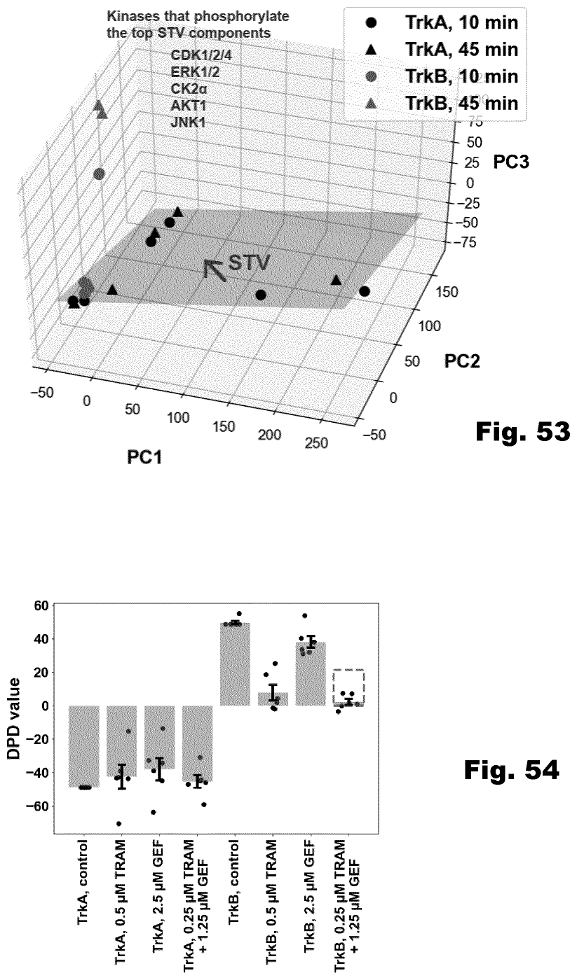

FIG. 53 shows the separation of MS phosphoproteomic patterns of TrkA and TrkB cell states and the STV projection into the PCA space. Following GF stimulation, TrkA and TrkB states were separated by a SVM. Projections of data points, the separating hyperplane and the STV (arrow) are shown in the space of the first three principal components. The text indicates the kinases that phosphorylate the top STV components.

FIG. 54 shows how ERBB and MEK inhibitors synergistically induce TrkB cell differentiation. The DPD values calculated using MS phosphoproteomics data for TrkA and TrkB cells treated with Trametinib (0.5 μM), Gefitinib (2.5 μM), and their combination (0.25 μM and 1.25 UM) at 45-minute stimulation. Data are presented as mean values+/−SEM for n=3 biologically independent samples examined over 2 independent experiments. Dashed bar shows the expected DPD value for the Bliss independence of a combination treatment of TrkB cells with Trametinib and Gefitinib.

Thus, cSTAR produces robust and reproducible results even when the input data differ vastly in scale and bias.

### RAF Inhibitor-Resistant Melanoma

To map drug resistance mechanisms, we applied cSTAR to an extensive RPPA dataset of 238 proteins measured under 89 perturbations of RAF inhibitor resistant SKMEL-133 cells. As different phenotypic states we selected proliferation (untreated cells) and apoptosis induced by combination treatment with MEK and PI3K/AKT/mTOR inhibitors.

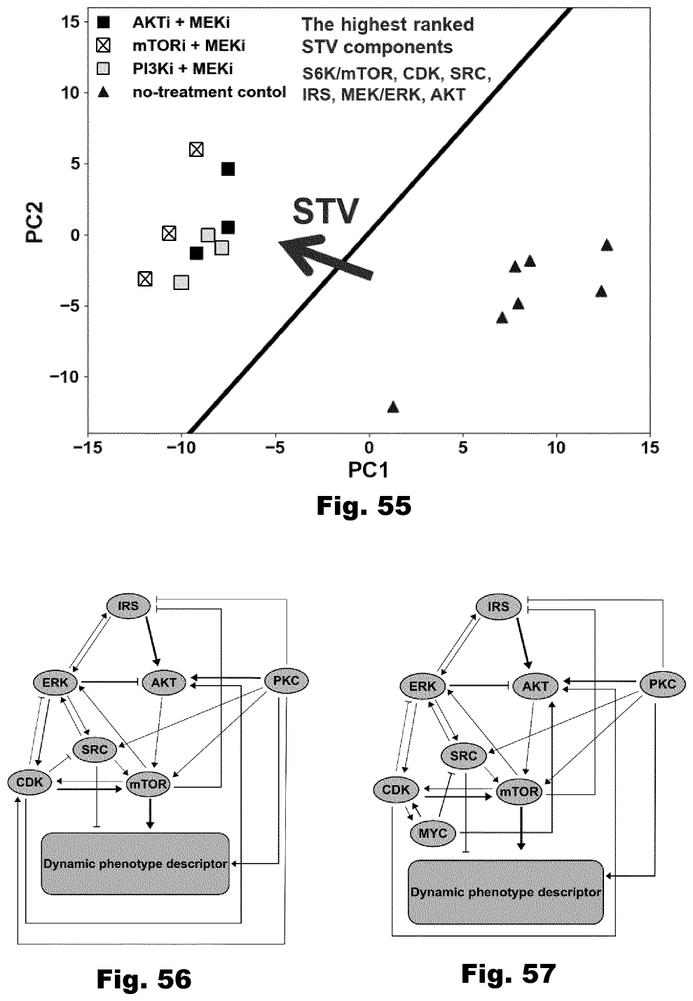

FIG. 55 shows the separation of apoptotic and proliferation states of SKMEL-133 cells and a projection into the PCA space. SVM separation of phosphoproteomic patterns of proliferation states in growing SKMEL-133 cells and apoptotic states after treatment with a combination of PI3K/AKT/mTOR and MEK inhibitors. The data are taken from Korkut, A. et al. “Perturbation biology nominates upstream-downstream drug combinations in RAF inhibitor resistant melanoma cells”. Elife 4, doi:10.7554/eLife.04640 (2015). Projections of the separated data points, the separating hyperplane (diagonal dividing line) and the STV (arrow) are shown in the space of the first two principal components.

The STV ranked the MEK/ERK, AKT, mTOR/S6K, SRC, CDK4/6, PKC, and IRS modules as the components of a core network that controlled these states. Next, we applied BMRA to single-drug perturbation data, inferring the core network circuitry and its connections to the DPD modules. The reconstructed network included known signaling routes, including the IRS-mediated activation of the ERK and AKT modules, AKT activation of mTOR, CDK4/6 activation by ERK and mTOR, and negative feedback from mTOR to IRS.

However, BMRA also uncovered activating connections from PKC to AKT, mTOR, SRC and CDK4/6, a negative connection from PKC to IRS, and CDK4/6-induced positive and negative feedback loops to the AKT and SRC modules.

FIG. 56 is an inferred topology of the core signaling network showing these features. Arrowheads indicate activation, blunt ends show inhibition, line widths indicate the absolute values of interaction strengths.

Based on their direct connections to the DPD, mTOR and PKC drive proliferation, while the phenotypical effect of other nodes is indirect. For instance, ERK activates mTOR through SRC and CDK4/6 to stimulate proliferation, partially counteracted by CDK4/6-mediated feedback inhibition of ERK. Although SRC directly inhibits the DPD, it stimulates proliferation on the systems level by activating mTOR.

The original publication by Korkut (referenced above in relation to FIG. 55) showed that MYC inhibition synergized with BRAF or MEK inhibition to suppress proliferation and induce apoptosis. Thus, we added MYC to our core network and re-inferred network connections.

FIG. 57 shows the inferred topology of the core signaling network with the addition of c-MYC. This extended network is very similar to the original network except that CDK inhibited SRC not directly but via MYC. The equivalence of these networks illustrates that BMRA allows zooming-in/out on the inferred connections by adding nodes of interest or deleting unimportant nodes.

Informed by the BMRA network reconstruction, we built a nonlinear dynamical model of SKMEL-133 cell signaling and phenotypic behavior. Because cSTAR enables building models of different granularities, we tested the effects of including or omitting MYC. Adding MYC only changed parameters of modules directly interacting with MYC without changing any model predictions. Thus, the ODE description of each network module can be extended to include additional mechanistic knowledge.

The model predicted that an mTOR inhibitor was the most efficient single drug to induce apoptosis in SKMEL-133 cells, whereas PI3K/AKT inhibition was less effective.

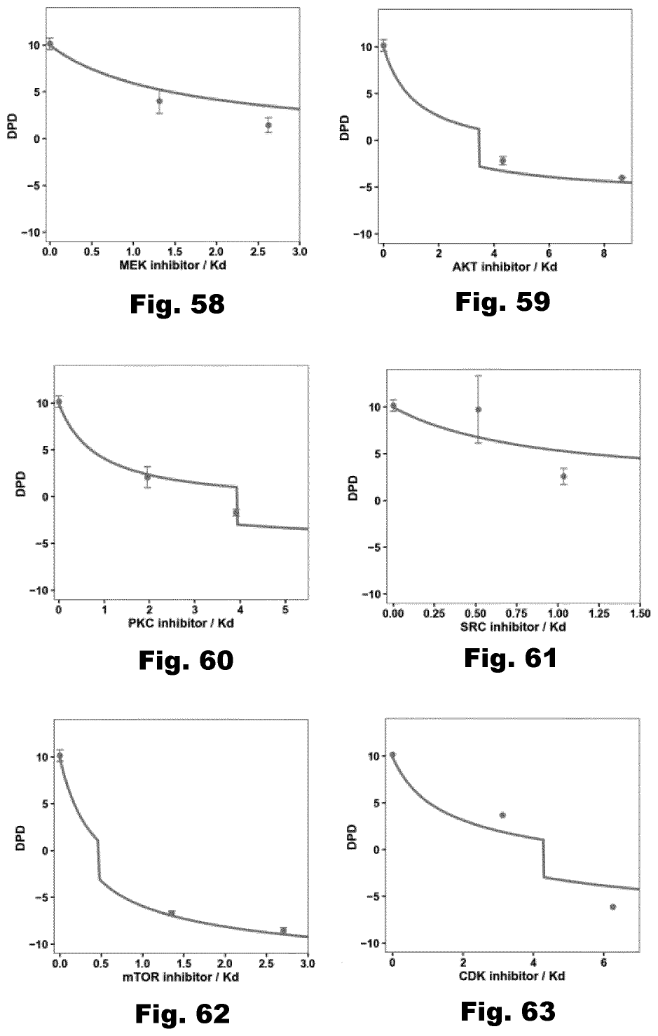

FIGS. 58-63 show the model calculated and experimentally determined DPD responses of SKMEL-133 cells to different inhibitors, i.e. respectively MEK, AKT, PKC, SRC, mTOR and CDK. The experimentally measured DPD values (dots) are calculated based on the data from the reference Korkut et al. Model-predicted (curves) DPD responses to many inhibitors exhibit abrupt DPD decreases at certain inhibitor doses caused by the loss of stability of a proliferation state and the induction of apoptosis in a threshold manner. Mathematically, an abrupt DPD decrease relates to a saddle-node bifurcation64 (a fold catastrophe) that occurs when a stable steady-state solution corresponding to a proliferation state disappears. Data are presented as mean values+/−SEM for n=3 biologically independent experiments.

This differential sensitivity is explained by the double-positive feedback between CDK4/6 and mTOR (FIGS. 56 & 57), which greatly increases the stimulation of proliferation by mTOR and CDK4/6. PKC inhibition also markedly reduced proliferation, as PKC directly influences the DPD, whereas inhibition of other nodes, including MEK/ERK signaling, was less effective.

The cSTAR model recapitulated the results by Korkut et al including the synergy between MEK and MYC inhibitors. Furthermore, the model predicted that combining Insulin/IGF1 receptor and PI3K/AKT inhibition enhances synergy. This result is supported by calculating the Talalay-Chou combination index and simulating SKMEL-133 cell maneuvering in Waddington's landscape following inhibitor treatments.

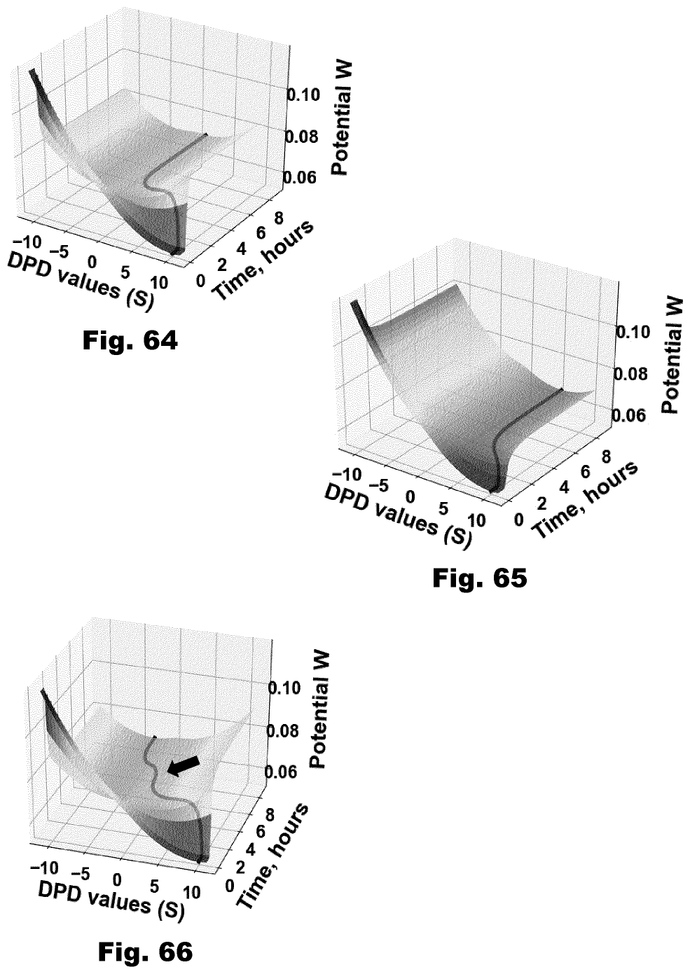

FIGS. 64-66 illustrate model-predicted SKMEL-133 cell maneuvering (shown as dark lines) in Waddington's landscape following inhibitor treatments. The Waddington landscape potential (W) is plotted against the DPD (S) and time. At t=0 cells reside in a highly proliferating state (high positive values of DPD). PI3K/AKT and Insulin/IGF1 receptor inhibitors were added at t=30 min at the 3K_d and 4K_d doses. When the inhibitors are applied separately (FIGS. 64 and 65), the decreasing DPD values remain in the proliferation region (positive DPD values). Treated with a combination of inhibitors in twice lower doses (1.5K_d for PI3K/AKT inhibitor and 2K_d for Insulin/IGF1 receptor inhibitor), the cells maneuver as shown in FIG. 66 to the apoptotic state manifested by negative DPD values. A threshold-like switch to negative DPD (black arrow) is a switch from proliferation to apoptosis.

Thus, PI3K/AKT or Insulin/IGF1 receptor inhibitors given separately do not switch the DPD to negative, apoptotic region (FIGS. 64 and 65). However, given in a combination at twice lower doses, they shift the DPD to apoptosis (FIG. 66). We also found that combining MEK/ERK and PI3K/AKT inhibitors was highly synergistic. This example shows that cSTAR is a powerful tool to analyze drug responses and predict synergistic combinations.

### Epithelial-Mesenchymal Transition (EMT)

cSTAR quantifies phenotypic changes via the DPD, opening the possibility to integrate different omics datasets by comparing the normalized DPD changes following perturbations. Testing this, we applied cSTAR to two datasets that analyzed EMT suppression by kinase inhibitors. One study (Cook, D. P. & Vanderhyden, B. C., “Context specificity of the EMT transcriptional response”. Nature Communications 11, 2142, doi:10.1038/s41467-020-16066-2 (2020)) used single-cell RNA sequencing (scRNA-seq) of four cancer cell lines stimulated with three different ligands, TGFβ, EGF and TNFα. The other (Chen, W. S. et al. “Uncovering axes of variation among single-cell cancer specimens”. Nature Methods 17, 302-310, doi:10.1038/s41592-019-0689-z (2020)) used single-cell resolution mass cytometry of phosphoproteomic responses in Py2T breast cancer cells stimulated with TGFβ.

The results of the cSTAR analysis correspond well to the original phenomenological observations and conclusions drawn in these papers. They show that cSTAR correctly captures the relationships between phenotypical and underlying molecular states. Moreover, cSTAR adds new insights. Interestingly, the DPD analysis of scRNAseq data demonstrated that at single-cell resolution the observed partial EMT states comprise a continuum of intermediate states between fully epithelial and fully mesenchymal states. To underpin these states with mechanistic interpretations, which was previously not possible, we applied BMRA to reconstruct the twelve signaling networks (four cell lines, three ligands), underlying these phenotypes in each cell type under each condition. These networks show how differential network topologies and connection strengths cause cell type and stimulation-specific responses. These reconstructions of different network topologies will help designing the most informative experiments to disentangle the relationships between these multiple EMT states.

MS proteomics data used in the experimental work are uploaded to the PRIDE database (accession number PXD028943). The RPPA data for SKMEL-133 cell line are available at http://projects.sanderlab.org/pertbio/. The CYTOF data for EMT in Py2T cell line32 is available at https://community.cytobank.org/cytobank/experiments#project-id=1296. Software code for the data analysis, network reconstruction and modeling are available at https://github.com/OleksiiR/cSTAR_Nature.

### Discussion

Cells employ signaling networks to process input signals and generate specific biological outputs. Signaling networks function via posttranslational modifications (PTMs) and are controlled by external cues and feedback loops mediated by PTMs and expression changes. Therefore, protein phosphorylation and expression datasets of cell responses to external cues contain rich information about cell states and fate decisions. There are several distinctive states, including differentiation, proliferation, senescence and apoptosis, which exhibit different phenotypes that can be well-detected by current experimental methods.

Omics data allow us to correlate cell-wide expression activity values with each phenotype, but how cell fate decisions are governed by signaling networks remains obscure.

Here, we have developed and experimentally validated the cSTAR approach that uses omics data to distinguish cell states, infer and quantify a core signaling network that determines transitions between these states.

This approach separates different cell states in the omics dataspace by machine learning methods and introduces the State Transition Vector (STV). Using the STV, the contributions of different protein abundances or activities to a cell state can be directly ranked. The components with high rank populate a core network, which drives global signaling patterns.

Subsequently, the causal core network connections and their strength are inferred using the Bayesian formulation of Modular Responses Analysis. It then builds mechanistic models to predict experimental perturbations that convert cellular states, e.g., proliferation into differentiation.

A key feature is that a process of cell fate decision making is included into a mechanistic model. We connected the signaling dynamics to the intuitively attractive picture of Waddington's landscape. Although many attempts were undertaken to quantify this landscape, the cell progression from one state to another was never connected to the responses of cell surface receptors and downstream signaling networks to external cues that drive this progression, and therefore experimental signaling activities data have not been used. Integrating biochemistry and physics the quantitative cSTAR approach determines how the activities of multiple signaling pathways dynamically control cell progression via Waddington's landscape, resulting in state transitions and fate decisions.

The cSTAR approach introduces a signaling driving force, which is coming from the responses of receptors and kinases to perturbations, such as external cues and pharmacological interventions, and is imposed on the initial potential, shaping Waddington's landscape. This force drives downstream signaling and transcription factor activities that ultimately determine cell fate decisions.

Because only omics data are available for this cell-wide signaling and transcription factor network, its mechanistic modeling is currently impractical. Previously, Waddington's landscape and transitions of stem cells to differentiation were interpreted by calculating multiple (quasi)steady states of a small transcription factor networks (Lu, M. et al. (2014). Construction of an Effective Landscape for Multistate Genetic Switches. Physical Review Letters 113, 078102).

A distinction of the cSTAR approach is the use of omics data obtained in response to experimental perturbations of core signaling network specified by the STV. Informed by these data, the cSTAR approach builds a core network mechanistic model, which includes global cell network as a dedicated module. The output of this module, a quantitative descriptor of cell phenotype, DPD together with the signaling pathway outputs are biochemically interpretable variables of the model. The model examines cell maneuvering in Waddington's landscape by monitoring the coordinated regulation of the components of the global cell network described by DPD. This model predicts how external and internal cues will change cell states.

The cSTAR approach can be flexibly extended to other omics datasets. If an omics dataset, for instance, RNAseq, contains data for only two different phenotypic states, a standard approach is determining of differentially expressed genes. Likewise, differentially phosphorylated phosphosites are determined for phosphoproteomics datasets. In the absence of perturbations, ranking of analytes by their contribution to the STV can provide similar information as above approaches. However, if a dataset contains at least a handful of perturbations, the calculation of a dot product of the STV and the perturbation vector helps us determine where each perturbation moves a cell state with respect to the state separation hyperplane, and thereby the change to the DPD brought about by this perturbation. Moreover, if an omics dataset contains a sufficient number of perturbations, the cSTAR approach determines (i) causal connections between signaling nodes of a core network driving cell fate decisions, (ii) connections to the DPD node linking signaling to cell state changes, (iii) nonlinear mechanistic model that predicts signaling and cell state responses to inhibitor perturbations.

Our application examples show that cSTAR can utilize and integrate diverse omics data including targeted and unbiased data of different scales as well as single cell data. This universality and scalability distinguishes cSTAR from other approaches that are more specialized in terms of input data, e.g. approaches relying on mRNA velocity input.

Summarizing, cSTAR offers a cell-specific mechanistic approach to describe, understand and purposefully manipulate cell fate decisions. As such it has numerous applications across biology that go beyond the use for interconverting proliferation and differentiation shown here as example.

1. A molecular evaluation method for predicting the molecular mechanisms through which a perturbation promotes or inhibits a cellular transition from a first cell state to a second cell state, comprising the operations:
a. processing data for a population of cells to identify cells associated with said first and second states respectively, wherein said processing comprises (i) mapping said cells in a multi-dimensional space whose dimensions correspond to distinct molecular features of the cells that define said cell states, the molecular features being selected from RNA expression, protein expression and posttranslational protein modification, and (ii) identifying clusters of cells in said representation associated with said first and second states;
b. constructing a hypersurface in said representation that separates said first and second states;
c. generating a state transition vector (STV) of unit length that determines the direction from the first state to the second state in said representation;
d. generating a ranked list of said molecular features, wherein each molecular feature is ranked by determining the magnitude of the STV component in a dimension associated with the molecular feature;
e. identifying within said ranked list the components of a core biochemical network comprising the top ranked components, or the components regulating the top ranked components, above a cut-off in the ranking;
f. calculating, in a reduced multi-dimensional space whose dimensions correspond to those molecular features not identified as components of the core biochemical network, a dimensionality-reduced representation of said hypersurface and a dimensionality-reduced STV;
g. determining the effect of a perturbation by generating a perturbation vector in the reduced multi-dimensional space, the perturbation vector connecting the centroids of point clouds associated with cell states before and after the perturbation was applied to cells;
h. classifying the perturbation as promoting a transition between said first and second cell states when the dot product of the perturbation vector and the dimensionality-reduced STV is positive or inhibiting said transition when said dot product is negative;
i. defining a Dynamic Phenotype Descriptor (DPD) that quantifies cell phenotypic changes in response to a perturbation by measuring whether the perturbation moves the cell states towards or away from the dimensionality-reduced hypersurface, wherein the DPD comprises the molecular features in the ranked list which are not components of the core biochemical network; and
j. calculating a respective causal network graph for each of the first and second cell states, wherein each causal network graph is a directed, weighted network graph having the same nodes, the nodes of each graph comprising each of the core components together with the DPD represented as a node, and each causal network graph having directed and weighted edges that are specific to the associated first or second cell state, whereby each of said graphs quantifies the effects of the core components on the DPD in the first or second cell state respectively and thereby describes the molecular mechanisms that characterise the first and second cell states.

2. The method of claim 1, wherein the operation of calculating a respective causal network graph comprises calculating a respective causal network connection matrix specifying the strength of connection between each of the core components and between each core component and the DPD.

3. The method of claim 2, wherein the calculation of a causal network connection matrix comprises inferring the topology and strengths of causal connections of the core network and the DPD using Modular Response Analysis.

4. The method of claim 3, wherein the Modular Response Analysis used is Bayesian Modular Response Analysis.

5. The method of claim 1, further comprising the operations of experimentally perturbing the cell states, observing the effect of the perturbation on the cell states, and inferring from the observed effects the strength of connection between each of the core components, and between each core component and the DPD.

6. The method of claim 5, wherein observing the effect of the perturbation on the cell states comprises measuring one or more molecular responses to the experimental perturbation.

7. The method of claim 5, wherein experimentally perturbing the cell states comprises applying a plurality of perturbations and observing the effect of the perturbations on the cell states.

8. The method of claim 1, wherein a perturbation comprises exposing cells to a chemical compound, exposing cells to a biological compound, inducing an epigenetic or genetic change in cells, exposing cells to pathogens, exposing cells to an interaction with other cells, and exposing cells to an interaction with a biological or artificial surface.

9. The method of claim 8, wherein
(i) the perturbation comprises exposing cells to a chemical compound, the chemical compound comprising a pharmaceutical drug, a toxin, or an environmental chemical;
(ii) the perturbation comprises exposing cells to a biological compound, the biological compound comprising a growth factor, a hormone, a cytokine, a toxin, an antibody, a cellular receptor, an siRNA, an shRNA, or a ligand;
(iii) the perturbation comprises exposing cells to an epigenetic or genetic change comprising an epigenetic modification, a gene mutation, a heterozygote gene knockout, a gene copy number aberration, a gene structural variant, or CRISPR/Cas9-mediated genetic modification; or
(iv) the perturbation comprises exposing cells to a pathogen comprising a virus or a bacterium.

10. The method of claim 1, wherein step (g) comprises processing data for a population of cells to which the or each perturbation has been applied, wherein said processing comprises (i) mapping said cells in said reduced multi-dimensional space, and (ii) identifying clusters of cells in said mapped cells associated with the cells before and after the perturbation is applied.

11. The method of claim 1, wherein identifying within said ranked list the components of a core biochemical network comprising the top ranked components above a cut-off in the ranking, comprises determining a cut-off in the ranking which maximises the number of components which can be mapped onto existing biochemical pathways while minimising the total number of ranked components used according to an optimisation function.

12. The method of claim 11, wherein determining the number of components which can be mapped onto existing biochemical pathways comprises determining from one or more databases whether each component can be mapped to a pathway whose characteristics are known from the one or more databases.

13. The method of claim 1, wherein said first and second cell states are any two states chosen from a set of three or more cell states, and wherein the step of processing data for a population of cells identifies cells associated with said three or more cell states by identifying clusters of cells in said representation associated with each of said three or more cell states.

14. The method of claim 1, wherein said hypersurface is a hyperplane.

15. The method of claim 1, wherein said distinct molecular features of the cells are identified in said processed data as a set of measured analyte levels each of which corresponds to a distinct molecular feature.

16. The method of claim 1, wherein said molecular features of the cells that define said cell states are selected from RNA expression, protein expression and posttranslational protein modification.

17. The method of claim 1, further comprising identifying an intervention likely to promote or inhibit a cellular transition between first and second cell states, by one or more of:
a. using the causal network graphs to identify an intervention that will change one cell state into another cell state; or
b. assessing by in silico simulations of kinetic computational models developed on the basis of said causal network graphs whether an intervention will move a said cell state along the STV away from, towards or across the separating hypersurface.

18. The method of claim 17, wherein the intervention is a combination of interventions, and the assessment in step (a) or (b) considers the effect of the interventions simultaneously.

19. The method of claim 17, wherein the intervention is a combination of interventions, and the assessment in step (a) or (b) considers the effect of the interventions serially.

20. The method of claim 17, wherein determining whether an intervention will change one cell state into another cell state comprises determining whether the distance from the first cell state data points to the hypersurface decreases following said intervention.

21. The method of claim 17, wherein determining whether an intervention will move a said cell state along the STV away from, towards or across the separating hypersurface comprises calculating a change in the DPD using a computational model built from the data, as given by:
$$\frac{dS}{dt} = f ( s ) + {\sum }_{j} {r}_{sj} ( \frac{{S}_{st . st .}}{{x}_{j}^{st . st}} ) {x}_{j} ( t )$$
wherein S is the DPD value being calculated by the model, ƒ(S) is the restoring driving force,
$${\sum }_{j} {r}_{sj} ( \frac{{S}_{st . st .}}{{x}_{j}^{st . st}} ) {x}_{j} ( t )$$
is the signaling driving force, x₁(t) are the outputs of signaling modules, r_{St} are the corresponding, BMRA-inferred connection coefficients to the STV, and s_{st.st.} and s_{ƒ}^{st.st} are the initial steady-state values of S and xⱼ before perturbations.
